(19) United States
(12) Patent Application Publication
(10) Pub. No.: US 2024/0115619 A1
(43) Pub. Date: Apr. 11, 2024

(54) TREATMENT OF DIABETES BY ENHANCEMENT OF PANCREATIC ISLET ENGRAFTMENT THROUGH REGENERATIVE IMMUNE MODULATION

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, NIceville, FL (US); Timothy WARBINGTON, Phoenix, AZ (US)

(73) Assignee: CREATIVE MEDICAL TECHNOLOGIES, INC., Phoenix, AZ (US)

(21) Appl. No.: 18/483,485

(22) Filed: Oct. 9, 2023

Related U.S. Application Data
(60) Provisional application No. 63/414,823, filed on Oct. 10, 2022.

(51) Int. Cl. A61K 35/28; A61K 35/39; A61K 39/00

(52) U.S. Cl. CPC A61K 35/28; A61K 35/39; A61K 39/001; A61K 39/4611; A61K 39/4622

(57) ABSTRACT

Disclosed are novel means of enhancing efficacy of pancreatic islet transplantation through administration of regenerative T cells concurrently with modification of the local microenvironment to provide a tolerogenic and angiogenic milieu. In one embodiment, the liver microenvironment is prepared for receipt of pancreatic islets by administration of tolerogenic cells antigen presenting cells, subsequently followed by administration of T cells and/or T regulatory cells conditioned by regenerative cells, and finally pancreatic islets are administered. In other embodiments devices are provided which allow for concurrent administration of pancreatic islets together with T cells and/or T regulatory cells that have been conditioned with regenerative cells. In another embodiment, the administration of T cells and/or T regulatory cells conditioned by regenerative cells is after administration of therapeutic pancreatic islets.

### CROSS-REFERENCE TO RELATED APPLICATIONS

This application claims priority to U.S. Provisional Application No. 63/414,823, titled “Treatment of Diabetes by Enhancement of Pancreatic Islet Engraftment Through Regenerative Immune Modulation”, and filed Oct. 10, 2022, which is incorporated herein by reference in its entirety.

### FIELD OF THE INVENTION

The invention pertains to the field of diabetes. More specifically, the invention pertains to treatment of diabetes through the administration of pancreatic islet grafts, whether autologous, xenogeneic, or in a preferred embodiment, allogeneic. The invention pertains to the field of increasing efficacy of engraftment and preventing generation of immunity towards the allograft in terms of rejection episodes. Furthermore, the invention provides means by which exogenous administration of cells increases the ability of the hepatic microenvironment to accept islet grafts.

### BACKGROUND OF THE INVENTION

Classical physiology teaches us that Insulin is a hypoglycemic hormone. Insulin dependent diabetes is caused by selective destruction of insulin-producing cells (islet β-cells) through immunological mechanisms, and its onset is known to result in hyperglycemia, which causes various disorders. In conventional therapy, preparations of the undersupplied insulin are administered (injected) to compensate for the shortage of insulin to redress hyperglycemia. However, strict blood sugar control is difficult in insulin-based therapies, which can lead to over-administration that can cause fatal hypoglycemia. After the onset of diabetes, diabetic vascular complications progress (such as retinopathy, nephropathy, and neuropathy). Conventional therapeutic methods such as insulin injections cannot halt this progress, leading to therapeutically serious problems. Blood sugar is physiologically controlled chiefly by the regulatory mechanism of islet β-cells; however, in insulin dependent diabetes, the deletion of these islets results in violent ups and downs in blood sugar level, causing the above-described clinical symptoms. In recent years, Europe and the United States have started clinical application of islet transplantation, where pancreatic islets of Langerhans (islets) are transplanted as a means for treating diabetes. This attempts treatment not by administering insulin, but by transplanting insulin-producing cells. The practical procedure of clinical islet transplantation is as follows: ultrasound-guided percutaneous, transhepatic portal vein catheterization is carried out under local anesthesia; and then donor islets are transplanted to the liver via the catheter. The islet grafts survive at the end of the portal vein, and control blood sugar level by secreting insulin. When successful, islet transplantation restores normal blood sugar to diabetic recipients, such that insulin treatment becomes unnecessary. To date, however, successful cases of clinical islet transplantation are limited. Further, transplantation to a single recipient requires islets isolated from the pancreas of two or three donors. Specifically, since islet function disorders that appear immediately after transplantation reduce graft viability, islet transplantation from a single donor to a single recipient is insufficient, and thus, transplantation from two to three donors to a single recipient is carried out. Some reports suggest only 20% to 30% of transplanted grafts survive. Details of these functional disorders remain unclear, but they pose an extremely serious problem in terms of improving the results of clinical islet transplantation.

As a novel transplantation therapy that can replace such pancreas transplantation, in recent years, pancreatic islet (also called Langerhans islet) transplantation technique has been energetically studied. The pancreatic islet means the group of endocrine cells that are scattered in an island shape in the tissue of the pancreas and contain β-cells secreting insulin. In addition, the pancreatic islet transplantation means a transplantation therapy in which pancreatic islet is separated from the pancreas offered from a donor to prepare a dispersion liquid of the pancreatic islet, and then the dispersion liquid is percutaneously administered to a patient. However, about 10 to 18 hours are required from pancreatectomy from a donor to preparation of pancreatic islet for the transplantation. Thus the prepared pancreatic islet must be preserved appropriately so that the pancreatic islet does not lose the vital force. In conventional methods, the preservation has been carried out by using such polystyrene tools as a flask or petri dish for culture. However, since many of the β-cells constituting pancreatic islet are deactivated or deadened around 24 hours after the separation and purification of pancreatic islet, it is found that there is temporal restriction from the preparation of the pancreatic islet to the completion of transplant operation. For example, there is such a case that the transplant operation cannot be carried out because the patient is not well enough to tolerate pancreatic islet transplantation once the donor islet cells are prepared, and the β-cells may be deactivated or deadened. Polystyrene petri dishes and flasks for culture conventionally used for a preservation container have a low oxygen permeability. A large amount of air was therefore required in the container, and only a small amount of pancreatic islet suspension prepared by suspending pancreatic islet could be put in the container. For example, a 100 mm petri dish can preserve only around 5 ml of a pancreatic islet suspension. Consequently, for preserving pancreatic islet separated from one pancreas, around 100 petri dishes has had to be used with a great deal of effort. In addition, when a petri dish or flask is used, pancreatic islet may adhere to the wall surface of the container, resulting in the loss of the pancreatic islet. Furthermore, containers made of polystyrene have a lid or a screw-type removable cap and have no sealing performance, so that the pancreatic islet may be contaminated with bacteria or viruses. If β-cells are deactivated, deadened, lost, and/or contaminated the will of the donor cannot be respected. Furthermore, at the time of pancreatic islet transplantation, pancreatic islet cells must be collected from each of petri dishes or flasks, and then temporarily preserved in a bag made of vinyl chloride and carried to an operation room. Concretely, the pancreatic islet liquid is aspirated from a petri dish or flask with a commercially available pipette or syringe, and then a port of the bag is punctured with a plastic needle or the like provided with a rubber co-injection part, through which the pancreatic islet liquid is slowly administered. However, when the preserved amount is large, the collection and injection process has to be repeated many times, which takes time and carries risks of bacterial contamination.

Conventionally, islet transplantation has been performed using islets isolated from the pancreases of brain-dead donors or donors under cardiac arrest. Recent reports also describe successful cases of living donor islet transplantation, in which islets are isolated and purified from a portion of pancreas excised from healthy donors and transplanted to diabetic patients. Such living donor islet transplantation is invasive and a burden for donors. Thus, it is preferable to enable treatments that suppress damage to transplanted islets just after transplantation and that use fewer donor islets. This also applies to transplantation of the partial or the entire pancreas.

### SUMMARY

Preferred embodiments are drawn to methods of increasing efficacy of an islet transplant for treatment of diabetes, wherein said method comprising the steps of: a) obtaining a diabetic patient in need of an islet transplant; b) administering to said patient one or more tolerogenic cell antigen presenting cells(s)/regenerative cell(s); c) administering to said patient one or more T cells and/or T regulatory cells that have been exposed to a regenerative cell for a sufficient time period to allow said T cells and/or T regulatory cells to possess ability to enhance islet engraftment; and d) administering to said patient islet cells/tissue/organ.

Preferred embodiments include methods wherein said regenerative cell has been in contact with said T cell and/or said regulatory T cell either in a cell to cell contact dependent manner or in contact through sharing of conditioned media.

Preferred embodiments include methods wherein said regenerative cells are mesenchymal stem cells derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; p) perinatal tissue

Preferred embodiments include methods wherein said mesenchymal stem cells express a marker or plurality of markers selected from a group comprising of: STRO-1, CD90, CD56, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Preferred embodiments include methods wherein said mesenchymal stem cells do not express substantial levels of HLA-DR, CD117, and CD45.

Preferred embodiments include methods wherein said mesenchymal stem cells are generated from a pluripotent stem cell.

Preferred embodiments include methods wherein said pluripotent stem cell is selected from a group comprising of: a) an embryonic stem cell; b) an inducible pluripotent stem cell; c) a parthenogenic stem cell; and d) a somatic cell nuclear transfer derived stem cell.

Preferred embodiments include methods wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

Preferred embodiments include methods wherein said inducible pluripotent stem cell possesses markers selected from a group comprising of: CD10, CD13, CD44, CD73, CD90, PDGFr-alpha, PD-L2, and HLA-A, -B, -C and possesses the ability to undergo at least 40 doublings in culture, while maintaining a normal karyotype upon passaging.

Preferred embodiments include methods wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

Preferred embodiments include methods wherein said somatic cell nuclear transfer derived stem cells possess a phenotype negative for SSEA-1 and positive for SSEA-3, SSEA-4, TRA-1-60, TRA-1-81, and alkaline phosphatase.

Preferred embodiments include methods wherein said mesenchymal stem cells are differentiated from a pluripotent stem cell source through culture in the presence of an inhibitor of the SMAD-2/3 pathway.

Preferred embodiments include methods wherein said mesenchymal stem cells are differentiated from a pluripotent stem cell source through culture in the presence of an inhibitor nucleic acid targeting the SMAD-2/3 pathway.

Preferred embodiments include methods wherein said nucleic acid inhibitor is selected from a group comprising of: a) an antisense oligonucleotide; b) a hairpin loop short interfering RNA; c) a chemically synthesized short interfering RNA molecule; and d) a hammerhead ribozyme.

Preferred embodiments include methods wherein said inhibitor of the SMAD-2/3 pathway is a small molecule inhibitor.

Preferred embodiments include methods wherein said small molecule inhibitor is SB-431542.

Preferred embodiments include methods wherein a selection process is used to enrich for mesenchymal stem cells differentiated from said pluripotent stem cell population.

Preferred embodiments include methods wherein said enrichment method comprises of positively selecting for cells expressing a marker associated with mesenchymal stem cells.

Preferred embodiments include methods wherein said marker of mesenchymal stem cells is selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Preferred embodiments include methods wherein the subject is known to have Type 1 Diabetes.

Preferred embodiments include methods wherein the subject has brittle diabetes that is resistant to optimal medical treatments.

Preferred embodiments include methods wherein the subject has had one or more prior hospitalizations for severe hypo- and/or hyper-glycemia including a state of ketosis.

Preferred embodiments include methods wherein the regenerative cells are prepared by administering to the subject an agent to mobilize regenerative cells from bone marrow into peripheral blood of the subject and isolating said regenerative cells from peripheral blood of the subject.

Preferred embodiments include methods wherein the agent to mobilize regenerative cells is granulocyte colony-stimulating factor (G-CSF).

Preferred embodiments include methods wherein the agent to mobilize regenerative cells is granulocyte monocyte colony-stimulating factor (GM-CSF).

Preferred embodiments include methods wherein the agent to mobilize regenerative cells is Leukemia Inhibiting Factor (LIF).

Preferred embodiments include methods wherein the agent to mobilize regenerative cells is HGF-1.

Preferred embodiments include methods wherein the agent to mobilize regenerative cells is FGF-1.

Preferred embodiments include methods wherein the agent to mobilize regenerative cells is FGF-2.

Preferred embodiments include methods wherein the agent to mobilize regenerative cells is AMD-3100.

Preferred embodiments include methods wherein the agent to mobilize regenerative cells is G-CSF/GM-CSF.

Preferred embodiments include methods wherein the agent to mobilize regenerative cells is ozone therapy.

Preferred embodiments include methods wherein the agent to mobilize regenerative cells is IL-2.

Preferred embodiments include methods wherein the agent to mobilize regenerative cells is FLT-3 ligand.

Preferred embodiments include methods wherein the agent to mobilize regenerative cells is TNF alpha.

Preferred embodiments include methods wherein the agent to mobilize regenerative cells is hCG.

Preferred embodiments include methods wherein the agent to mobilize regenerative cells is hyperbaric oxygen.

Preferred embodiments include methods wherein the agent to mobilize regenerative cells is BDNF.

Preferred embodiments include methods wherein the agent to mobilize regenerative cells is NGF-1.

Preferred embodiments include methods wherein the agent to mobilize regenerative cells is VEGF.

Preferred embodiments include methods wherein the regenerative cells are isolated from peripheral circulation of the subject by apheresis using an antibody that has selective affinity to said regenerative cells.

Preferred embodiments include methods wherein said regenerative cells are isolated with an antibody capable of binding cells expressing CD31.

Preferred embodiments include methods wherein said regenerative cells are isolated with an antibody capable of binding cells expressing CD33.

Preferred embodiments include methods wherein said regenerative cells are isolated with an antibody capable of binding cells expressing CD34.

Preferred embodiments include methods wherein said regenerative cells are isolated with an antibody capable of binding cells expressing CD133.

Preferred embodiments include methods wherein said regenerative cells are isolated with an antibody capable of binding cells expressing CD39.

Preferred embodiments include methods wherein said regenerative cells are isolated with an antibody capable of binding cells expressing CD31 and CD34.

Preferred embodiments include methods wherein said regenerative cells are isolated with an antibody capable of binding cells expressing CD31 and CD33.

Preferred embodiments include methods wherein said regenerative cells are isolated with an antibody capable of binding cells expressing CD31 and VEGF receptor.

Preferred embodiments include methods wherein said regenerative cells are isolated with an antibody capable of binding cells expressing CD31 and HGF-1 receptor.

Preferred embodiments include methods wherein said regenerative cells are isolated with an antibody capable of binding cells expressing CD31 and stem cell factor.

Preferred embodiments include methods wherein said regenerative cells are isolated with an antibody capable of binding cells expressing TLR-4 and CD34.

Preferred embodiments include methods wherein said regenerative cells are isolated with an antibody capable of binding cells expressing TLR-4 and CD33.

Preferred embodiments include methods wherein said regenerative cells are isolated with an antibody capable of binding cells expressing TLR-4 and VEGF receptor.

Preferred embodiments include methods wherein said regenerative cells are isolated with an antibody capable of binding cells expressing TLR-4 and HGF-1 receptor.

Preferred embodiments include methods wherein said regenerative cells are isolated with an antibody capable of binding cells expressing TLR-4 and stem cell factor.

Preferred embodiments include methods wherein said regenerative cells are isolated with an antibody capable of binding cells expressing TLR-4 and CD90.

Preferred embodiments include methods wherein said regenerative cells are isolated with an antibody capable of binding cells expressing TLR-4 and CD13.

Preferred embodiments include methods wherein said regenerative cells are isolated with an antibody capable of binding cells expressing TLR-4 and CD29.

Preferred embodiments include methods wherein said regenerative cells are isolated with an antibody capable of binding cells expressing TLR-4 and CD44.

Preferred embodiments include methods wherein said regenerative cells are isolated with an antibody capable of binding cells expressing TLR-4 and CD71.

Preferred embodiments include methods wherein said regenerative cells are isolated with an antibody capable of binding cells expressing TLR-4 and CD73.

Preferred embodiments include methods wherein said regenerative cells are isolated with an antibody capable of binding cells expressing TLR-4 and CD105.

Preferred embodiments include methods wherein said regenerative cells are isolated with an antibody capable of binding cells expressing TLR-4 and CD166.

Preferred embodiments include methods wherein said regenerative cells are isolated with an antibody capable of binding cells expressing TLR-4 and STRO-1.

Preferred embodiments include methods wherein said regenerative cells are isolated with an antibody capable of binding cells expressing TLR-4 and STRO-4.

Preferred embodiments include methods wherein said regenerative cells are isolated with an antibody capable of binding cells expressing TLR-4 and TNF receptor p55.

Preferred embodiments include methods wherein said regenerative cells are isolated with an antibody capable of binding cells expressing TLR-4 and TNF receptor p75.

Preferred embodiments include methods wherein said regenerative cells are isolated with an antibody capable of binding cells expressing TLR-4 and CD227.

Preferred embodiments include methods wherein said regenerative cells are isolated with an antibody capable of binding cells expressing c-kit and CD34.

Preferred embodiments include methods wherein said regenerative cells are isolated with an antibody capable of binding cells expressing c-kit and CD33.

Preferred embodiments include methods wherein said regenerative cells are isolated with an antibody capable of binding cells expressing c-kit and VEGF receptor.

Preferred embodiments include methods wherein said regenerative cells are isolated with an antibody capable of binding cells expressing c-kit and HGF-1 receptor.

Preferred embodiments include methods wherein said regenerative cells are isolated with an antibody capable of binding cells expressing c-kit and stem cell factor.

Preferred embodiments include methods wherein said regenerative cells are isolated with an antibody capable of binding cells expressing c-kit and CD90.

Preferred embodiments include methods wherein said regenerative cells are isolated with an antibody capable of binding cells expressing c-kit and CD13.

Preferred embodiments include methods wherein said regenerative cells are isolated with an antibody capable of binding cells expressing c-kit and CD29.

Preferred embodiments include methods wherein said regenerative cells are isolated with an antibody capable of binding cells expressing c-kit and CD44.

Preferred embodiments include methods wherein said regenerative cells are isolated with an antibody capable of binding cells expressing c-kit and CD71.

Preferred embodiments include methods wherein said regenerative cells are isolated with an antibody capable of binding cells expressing c-kit and CD73.

Preferred embodiments include methods wherein said regenerative cells are isolated with an antibody capable of binding cells expressing c-kit and CD105.

Preferred embodiments include methods wherein said regenerative cells are isolated with an antibody capable of binding cells expressing c-kit and CD166.

Preferred embodiments include methods wherein said regenerative cells are isolated with an antibody capable of binding cells expressing c-kit and STRO-1.

Preferred embodiments include methods wherein said regenerative cells are isolated with an antibody capable of binding cells expressing c-kit and STRO-4.

Preferred embodiments include methods wherein said regenerative cells are isolated with an antibody capable of binding cells expressing c-kit and TNF receptor p55.

Preferred embodiments include methods wherein said regenerative cells are isolated with an antibody capable of binding cells expressing c-kit and TNF receptor p75.

Preferred embodiments include methods wherein said regenerative cells are isolated with an antibody capable of binding cells expressing c-kit and CD227.

Preferred embodiments include methods wherein said regenerative cells are isolated with an antibody capable of binding cells expressing OCT-4 and CD34.

Preferred embodiments include methods wherein said regenerative cells are isolated with an antibody capable of binding cells expressing OCT-4 and CD33.

Preferred embodiments include methods wherein said regenerative cells are isolated with an antibody capable of binding cells expressing OCT-4 and VEGF receptor.

Preferred embodiments include methods wherein said regenerative cells are isolated with an antibody capable of binding cells expressing OCT-4 and HGF-1 receptor.

Preferred embodiments include methods wherein said regenerative cells are isolated with an antibody capable of binding cells expressing OCT-4 and stem cell factor.

Preferred embodiments include methods wherein said regenerative cells are isolated with an antibody capable of binding cells expressing OCT-4 and CD90.

Preferred embodiments include methods wherein said regenerative cells are isolated with an antibody capable of binding cells expressing OCT-4 and CD13.

Preferred embodiments include methods wherein said regenerative cells are isolated with an antibody capable of binding cells expressing OCT-4 and CD29.

Preferred embodiments include methods wherein said regenerative cells are isolated with an antibody capable of binding cells expressing OCT-4 and CD44.

Preferred embodiments include methods wherein said regenerative cells are isolated with an antibody capable of binding cells expressing OCT-4 and CD71.

Preferred embodiments include methods wherein said regenerative cells are isolated with an antibody capable of binding cells expressing OCT-4 and CD73.

Preferred embodiments include methods of preventing or treating rejection of allogenic/xenogenic cell/tissue/organ implantation for diabetes by administering regenerative cells combined with immunosuppressive agents.

Preferred embodiments include methods wherein said regenerative cells are mesenchymal stem cells.

Preferred embodiments include methods wherein said mesenchymal stem cells are naturally occurring mesenchymal stem cells.

Preferred embodiments include methods wherein said mesenchymal stem cells are generated in vitro.

Preferred embodiments include methods wherein said naturally occurring mesenchymal stem cells are tissue derived.

Preferred embodiments include methods wherein said naturally occurring mesenchymal stem cells are derived from a bodily fluid.

Preferred embodiments include methods wherein said tissue derived mesenchymal stem cells are selected from a group comprising of: a) bone marrow; b) perivascular tissue; c) adipose tissue; d) perinatal tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; and v) skeletal muscle tissue.

Preferred embodiments include methods wherein said tissue derived mesenchymal stem cells are isolated from tissues containing cells selected from a group of cells comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Preferred embodiments include methods wherein said mesenchymal stem cells are plastic adherent.

Preferred embodiments include methods wherein said mesenchymal stem cells express a marker selected from a group comprising of: a) CD73; b) CD90; and c) CD105.

Preferred embodiments include methods wherein said mesenchymal stem cells lack expression of a marker selected from a group comprising of: a) CD14; b) CD45; and c) CD34.

Preferred embodiments include methods wherein said mesenchymal stem cells from perinatal tissue express markers selected from a group comprising of: a) oxidized low density lipoprotein receptor 1; b) chemokine receptor ligand 3; and c) granulocyte chemotactic protein.

Preferred embodiments include methods wherein said mesenchymal stem cells from perinatal tissue do not express markers selected from a group comprising of: a) CD117; b) CD31; c) CD34; and CD45.

Preferred embodiments include methods wherein said mesenchymal stem cells from perinatal tissue express, relative to a human fibroblast, increased levels of interleukin 8 and reticulon 1.

Preferred embodiments include methods wherein said mesenchymal stem cells from perinatal tissue have the potential to differentiate into cells of at least a skeletal muscle, vascular smooth muscle, pericyte or vascular endothelium phenotype.

Preferred embodiments include methods wherein said mesenchymal stem cells from perinatal tissue express markers selected from a group comprising of: a) CD10; b) CD13; c) CD44; d) CD73; and e) CD90.

Preferred embodiments include methods wherein said perinatal tissue stem cell is an isolated umbilical cord tissue substantially free of blood that is capable of self-renewal and expansion in culture.

Preferred embodiments include methods wherein said perinatal tissue stem cells have the potential to differentiate into cells of other phenotypes.

Preferred embodiments include methods wherein said other phenotypes comprise: a) osteocytic; b) adipogenic; and c) chondrogenic differentiation.

Preferred embodiments include methods wherein said perinatal tissue stem cells can undergo at least 20 doublings in culture.

Preferred embodiments include methods wherein said perinatal tissue stem cell maintains a normal karyotype upon passaging.

Preferred embodiments include methods wherein said perinatal tissue stem cell expresses a marker selected from a group of markers comprised of: a) CD10; b) CD13; c) CD44; d) CD73; e) CD90; f) PDGFr-alpha; g) PD-L2; and h) HLA-A, -B, -C.

Preferred embodiments include methods wherein said perinatal tissue stem cells do not express one or more markers selected from a group comprising of: a) CD31; b) CD34; c) CD45; d) CD80; e) CD86; 0 CD117; g) CD141; h) CD178; i) B7-H2; j) HLA-G; and k) HLA-DR, DP, DQ.

Preferred embodiments include methods wherein said perinatal tissue cells secrete factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; l) RANTES; and m) TIMP1.

Preferred embodiments include methods wherein said perinatal tissue cells express markers selected from a group comprising of: a) TRA1-60; b) TRA1-81; c) SSEA3; and d) SSEA4.

Preferred embodiments include methods wherein said perinatal tissue cells are positive for alkaline phosphatase staining.

Preferred embodiments include methods wherein said perinatal tissue cells are capable of differentiating into one or more lineages selected from a group comprising of: a) ectoderm; b) mesoderm, and; c) endoderm.

Preferred embodiments include methods wherein said bone marrow derived mesenchymal stem cells possess markers selected from a group comprising of: a) CD73; b) CD90; and c) CD105.

Preferred embodiments include methods wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; l) 6-19; m) thrombomodulin; n) telomerase; o) CD10; p) CD13; and q) integrin beta.

Preferred embodiments include methods wherein said bone marrow derived mesenchymal stem cell is a mesenchymal stem cell progenitor cell.

Preferred embodiments include methods wherein said mesenchymal progenitor cells are a population of bone marrow mesenchymal stem cells enriched for cells containing STRO-1.

Preferred embodiments include methods wherein said mesenchymal progenitor cells express both STRO-1 and VCAM-1.

Preferred embodiments include methods wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.

Preferred embodiments include methods wherein said bone marrow mesenchymal stem cells lack expression of CD14, CD34, and CD45.

Preferred embodiments include methods wherein said STRO-1 expressing cells are positive for a marker selected from a group comprising of: a) VACM-1; b) TKY-1; c) CD146 and; d) STRO-2.

Preferred embodiments include methods wherein said bone marrow mesenchymal stem cell express markers selected from a group comprising of: a) CD13; b) CD34; c) CD56 and; d) CD117.

Preferred embodiments include methods wherein said bone marrow mesenchymal stem cells do not express CD10.

Preferred embodiments include methods wherein said bone marrow mesenchymal stem cells do not express CD2, CD5, CD14, CD19, CD33, CD45, and DRII.

Preferred embodiments include methods wherein said bone marrow mesenchymal stem cells express CD13, CD34, CD56, CD90, CD117 and nestin, and which do not express CD2, CD3, CD10, CD14, CD16, CD31, CD33, CD45 and CD64.

Preferred embodiments include methods wherein said skeletal muscle stem cells express markers selected from a group comprising of: a) CD13; b) CD34; c) CD56 and; d) CD117.

Preferred embodiments include methods wherein said skeletal muscle mesenchymal stem cells do not express CD10.

Preferred embodiments include methods wherein said skeletal muscle mesenchymal stem cells do not express CD2, CD5, CD14, CD19, CD33, CD45, and DRII.

Preferred embodiments include methods wherein said bone marrow mesenchymal stem cells express CD13, CD34, CD56, CD90, CD117 and nestin, and which do not express CD2, CD3, CD10, CD14, CD16, CD31, CD33, CD45 and CD64.

Preferred embodiments include methods wherein said perinatal tissue stem cells possess markers selected from a group comprising of: a) CD29; b) CD73; c) CD90; d) CD166; e) SSEA4; f) CD9; g) CD44; h) CD146; and i) CD105.

Preferred embodiments include methods wherein said perinatal tissue stem cells do not express markers selected from a group comprising of: a) CD45; b) CD34; c) CD14; d) CD79; e) CD106; f) CD86; g) CD80; h) CD19; i) CD117; j) Stro-1; and k) HLA-DR.

Preferred embodiments include methods wherein said perinatal tissue stem cells express CD29, CD73, CD90, CD166, SSEA4, CD9, CD44, CD146, and CD105.

Preferred embodiments include methods wherein said perinatal tissue stem cells do not express CD45, CD34, CD14, CD79, CD106, CD86, CD80, CD19, CD117, Stro-1, and HLA-DR.

Preferred embodiments include methods wherein said perinatal tissue stem cells are positive for SOX2.

Preferred embodiments include methods wherein said perinatal tissue stem cells are positive for OCT4.

Preferred embodiments include methods wherein said perinatal tissue stem cells are positive for OCT4 and SOX2.

Preferred embodiments include methods wherein said tolerogenic cells are monocytes.

Preferred embodiments include methods wherein said monocytes are capable of inducing generation of T regulatory (Treg) cells.

Preferred embodiments include methods wherein said Treg cells are capable of suppressing rejection of allogeneic islet cells.

Preferred embodiments include methods wherein said Treg cells are capable of suppressing dendritic cell maturation.

Preferred embodiments include methods wherein said Treg cells are capable of suppressing neutrophil activation.

Preferred embodiments include methods wherein said Treg cells are capable of suppressing T cell proliferation.

Preferred embodiments include methods wherein said Treg cells are capable of suppressing T cell cytotoxicity.

Preferred embodiments include methods wherein said Treg cells are capable of suppressing T cell interferon gamma production.

Preferred embodiments include methods wherein said Treg cells are capable of suppressing T cell interleukin-2 production.

Preferred embodiments include methods wherein said Treg cells are capable of suppressing T cell interleukin-12 production.

Preferred embodiments include methods wherein said Treg cells are capable of suppressing T cell interleukin-17 production.

Preferred embodiments include methods wherein said Treg cells are capable of suppressing T cell interleukin-21 production.

Preferred embodiments include methods wherein said Treg cells are capable of suppressing T cell interleukin-18 production.

Preferred embodiments include methods wherein said Treg cells are capable of enhancing T cell interleukin-4 production.

Preferred embodiments include methods wherein said Treg cells are capable of enhancing T cell interleukin-13 production.

Preferred embodiments include methods wherein said Treg cells are capable of enhancing T cell interleukin-9 production.

Preferred embodiments include methods wherein said Treg cells are capable of enhancing T cell interleukin-10 production.

Preferred embodiments include methods wherein said Treg cells are capable of enhancing T cell interleukin-38 production.

Preferred embodiments include methods wherein said Treg cells are capable of enhancing T cell interleukin-35 production.

Preferred embodiments include methods wherein said Treg cells are capable of enhancing T cell TGF-beta production.

Preferred embodiments include methods wherein said Treg cells are capable of enhancing T cell interferon alpha production.

Preferred embodiments include methods wherein said Treg cells are capable of enhancing T cell interferon beta production.

Preferred embodiments include methods wherein said monocytes are alternatively activated.

Preferred embodiments include methods wherein said monocytes are treated with interleukin-4 for a sufficient time and at a sufficient concentration to suppress ability to activate NF-kappa B in response to ligation of toll like receptor 3.

Preferred embodiments include methods wherein said monocytes are treated with interleukin-4 for a sufficient time and at a sufficient concentration to suppress ability to activate NF-kappa B in response to ligation of toll like receptor 4.

Preferred embodiments include methods wherein said monocytes are treated with interleukin-4 for a sufficient time and at a sufficient concentration to suppress ability to activate NF-kappa B in response to ligation of toll like receptor 9.

Preferred embodiments include methods wherein said monocytes are treated with interleukin-10 for a sufficient time and at a sufficient concentration to suppress ability to activate NF-kappa B in response to ligation of toll like receptor 3.

Preferred embodiments include methods wherein said monocytes are treated with interleukin-10 for a sufficient time and at a sufficient concentration to suppress ability to activate NF-kappa B in response to ligation of toll like receptor 4.

Preferred embodiments include methods wherein said monocytes are treated with interleukin-10 for a sufficient time and at a sufficient concentration to suppress ability to activate NF-kappa B in response to ligation of toll like receptor 9.

Preferred embodiments include methods wherein said monocytes are treated with interleukin-13 for a sufficient time and at a sufficient concentration to suppress ability to activate NF-kappa B in response to ligation of toll like receptor 3.

Preferred embodiments include methods wherein said monocytes are treated with interleukin-13 for a sufficient time and at a sufficient concentration to suppress ability to activate NF-kappa B in response to ligation of toll like receptor 4.

Preferred embodiments include methods wherein said monocytes are treated with interleukin-13 for a sufficient time and at a sufficient concentration to suppress ability to activate NF-kappa B in response to ligation of toll like receptor 9.

Preferred embodiments include methods wherein said monocytes are treated with interleukin-16 for a sufficient time and at a sufficient concentration to suppress ability to activate NF-kappa B in response to ligation of toll like receptor 3.

Preferred embodiments include methods wherein said monocytes are treated with interleukin-16 for a sufficient time and at a sufficient concentration to suppress ability to activate NF-kappa B in response to ligation of toll like receptor 4.

Preferred embodiments include methods wherein said monocytes are treated with interleukin-16 for a sufficient time and at a sufficient concentration to suppress ability to activate NF-kappa B in response to ligation of toll like receptor 9.

Preferred embodiments include methods wherein said monocytes are treated with interleukin-20 for a sufficient time and at a sufficient concentration to suppress ability to activate NF-kappa B in response to ligation of toll like receptor 3.

Preferred embodiments include methods wherein said monocytes are treated with interleukin-20 for a sufficient time and at a sufficient concentration to suppress ability to activate NF-kappa B in response to ligation of toll like receptor 4.

Preferred embodiments include methods wherein said monocytes are treated with interleukin-20 for a sufficient time and at a sufficient concentration to suppress ability to activate NF-kappa B in response to ligation of toll like receptor 9.

Preferred embodiments include methods wherein said monocytes are treated with interleukin-27 for a sufficient time and at a sufficient concentration to suppress ability to activate NF-kappa B in response to ligation of toll like receptor 3.

Preferred embodiments include methods wherein said monocytes are treated with interleukin-27 for a sufficient time and at a sufficient concentration to suppress ability to activate NF-kappa B in response to ligation of toll like receptor 4.

Preferred embodiments include methods wherein said monocytes are treated with interleukin-27 for a sufficient time and at a sufficient concentration to suppress ability to activate NF-kappa B in response to ligation of toll like receptor 9.

Preferred embodiments include methods wherein said monocytes are treated with interleukin-35 for a sufficient time and at a sufficient concentration to suppress ability to activate NF-kappa B in response to ligation of toll like receptor 3.

Preferred embodiments include methods wherein said monocytes are treated with interleukin-35 for a sufficient time and at a sufficient concentration to suppress ability to activate NF-kappa B in response to ligation of toll like receptor 4.

Preferred embodiments include methods wherein said monocytes are treated with interleukin-35 for a sufficient time and at a sufficient concentration to suppress ability to activate NF-kappa B in response to ligation of toll like receptor 9.

Preferred embodiments include methods wherein said monocytes are treated with interleukin-38 for a sufficient time and at a sufficient concentration to suppress ability to activate NF-kappa B in response to ligation of toll like receptor 3.

Preferred embodiments include methods wherein said monocytes are treated with interleukin-38 for a sufficient time and at a sufficient concentration to suppress ability to activate NF-kappa B in response to ligation of toll like receptor 4.

Preferred embodiments include methods wherein said monocytes are treated with interleukin-38 for a sufficient time and at a sufficient concentration to suppress ability to activate NF-kappa B in response to ligation of toll like receptor 9.

Preferred embodiments include methods wherein said monocytes are treated with TGF-beta for a sufficient time and at a sufficient concentration to suppress ability to activate NF-kappa B in response to ligation of toll like receptor 3.

Preferred embodiments include methods wherein said monocytes are treated with TGF-beta for a sufficient time and at a sufficient concentration to suppress ability to activate NF-kappa B in response to ligation of toll like receptor 4.

Preferred embodiments include methods wherein said monocytes are treated with TGF-beta for a sufficient time and at a sufficient concentration to suppress ability to activate NF-kappa B in response to ligation of toll like receptor 9.

Preferred embodiments include methods of increasing efficacy of an islet transplant for treatment of diabetes, wherein said method comprising the steps of: a) obtaining a diabetic patient in need of an islet (cell/tissue/organ) transplant; b) administering to said patient islet cells (auto/allo/xeno), c) administering to said patient one or more T cells and/or T regulatory cells that have been exposed to a regenerative cell for a sufficient time period to allow said T cells and/or T regulatory cells to possess ability to enhance islet engraftment (cell/tissue/organ); and d) to decrease rejection.

The method in claim 200, wherein generating the T cell possessing the ability to suppress activation of another T cell while concurrently having the ability to stimulate regenerative processes.

Preferred embodiments include methods wherein said T cell possesses the marker CD4.

Preferred embodiments include methods wherein said T cell is thymic derived.

Preferred embodiments include methods wherein said T cell is derived from a pluripotent stem cell.

Preferred embodiments include methods wherein said T cell expresses the transcription factor FoxP3.

Preferred embodiments include methods wherein said suppression of another T cell means inhibition of T cell proliferation.

Preferred embodiments include methods wherein said T cell proliferation is induced by an agent selected from one or more agents from a group comprising of: a) a mitogen; b) a cytokine; c) a molecule capable of activating aT cell receptor in an antigen nonspecific manner; and d) a molecule capable of activating aT cell receptor in an antigen specific manner.

Preferred embodiments include methods wherein said mitogen is conconavalin-A, phytohemagluttinin, pokeweed mitogen, Galanthus nivalis Lectin, or Cytokine interleukin-1 beta, interleukin-2, interleukin-3, interleukin-4, interleukin-7, interleukin-10, interleukin-12, interleukin-18, interleukin-17, interleukin-20, interleukin-35, interferon alpha, interferon beta, interferon gamma, interferon omega.

Preferred embodiments include methods wherein said suppression of another T cell means inhibition of T cell cytotoxic activity.

Preferred embodiments include methods wherein said T cell cytotoxic activity is mediated by Fas Ligand.

Preferred embodiments include methods wherein said T cell cytotoxic activity is mediated by TRAIL.

Preferred embodiments include methods wherein said T cell cytotoxic activity is mediated by perforin.

Preferred embodiments include methods wherein said T cell cytotoxic activity is mediated by granzyme A.

Preferred embodiments include methods wherein said T cell cytotoxic activity is mediated by granzyme B.

Preferred embodiments include methods wherein said T cell cytotoxic activity is mediated by TNF-alpha.

Preferred embodiments include methods wherein said T cell cytotoxic activity is mediated by TNF-beta.

Preferred embodiments include methods wherein said T cell cytotoxic activity is mediated by induction of apoptosis.

Preferred embodiments include methods wherein said T cell cytotoxic activity is mediated by induction of necrosis.

Preferred embodiments include methods wherein said T cell cytotoxic activity is mediated by pyroptosis.

Preferred embodiments include methods wherein said T cell cytotoxic activity is mediated by necroptosis.

Preferred embodiments include methods wherein said T cell cytotoxic activity is mediated by alternation in mitochondrial membrane potential in the target cell.

Preferred embodiments include methods wherein said suppression of another T cell is downregulation of cytokine production of the target cell.

Preferred embodiments include methods wherein said cytokine production is induced by an agent selected from one or more agents from a group comprising of: a) a mitogen; b) a cytokine; c) a molecule capable of activating aT cell receptor in an antigen nonspecific manner; and d) a molecule capable of activating aT cell receptor in an antigen specific manner.

Preferred embodiments include methods mitogen is conconavalin-A, phytohemagluttinin, pokeweed mitogen, Galanthus nivalis Lectin, or Cytokine interleukin-1 beta, interleukin-2, interleukin-3, interleukin-4, interleukin-7, interleukin-10, interleukin-12, interleukin-18, interleukin-17, interleukin-20, interleukin-35, interferon alpha, interferon beta, interferon gamma, interferon omega.

Preferred embodiments include methods wherein said molecule capable of activating said TCR in an antigen non-specific manner is an antibody.

Preferred embodiments include methods wherein said antibody is capable of ligating TCR.

Preferred embodiments include methods wherein said antibody is capable of ligating CD3.

Preferred embodiments include methods wherein said antibody is OKT3.

Preferred embodiments include methods wherein said molecule capable of activating said TCR in an antigen non-specific manner is a microbody.

Preferred embodiments include methods wherein said molecule capable of activating said TCR in an antigen non-specific manner is a bispecific antibody.

Preferred embodiments include methods wherein said molecule capable of activating said TCR in an antigen non-specific manner is a recombinant protein.

Preferred embodiments include methods wherein said molecule capable of activating said TCR in an antigen non-specific manner is an aptamer.

Preferred embodiments include methods wherein said molecule capable of activating said TCR in an antigen non-specific manner is a DNano particle.

Preferred embodiments include methods wherein said molecule capable of activating said TCR in an antigen specific manner is an autoantigen.

Preferred embodiments include methods wherein said regenerative properties are ability to stimulate angiogenesis.

Preferred embodiments include methods wherein said angiogenesis is creation of collateral circulation around the pancreatic islets and transplanted tissue.

Preferred embodiments include methods wherein said collateral circulation is used to treat ischemic conditions.

Preferred embodiments include methods wherein said ischemic conditions are associated with activation of hypoxia inducible factor-1.

Preferred embodiments include methods wherein said angiogenesis is associated with production of VEGF.

Preferred embodiments include methods wherein said angiogenesis is associated with mobilization of endothelial progenitor cells.

Preferred embodiments include methods wherein said angiogenesis is associated with mobilization of mesenchymal stem cells.

Preferred embodiments include methods wherein said angiogenesis is associated with mobilization of hematopoietic stem cells.

Preferred embodiments include methods wherein said T cells are extracted from a source selected from a group of sources comprising of: a) peripheral blood; b) mobilized peripheral blood; c) umbilical cord blood; d) menstrual blood; e) bone marrow; f) cerebral spinal fluid; and g) adipose tissue.

Preferred embodiments include methods wherein said T cell is a CD4 T cell.

Preferred embodiments include methods wherein said T cell obtained by culture of peripheral blood mononuclear cells in the presence of conditioned/enhanced media from an activated mesenchymal stem cell.

Preferred embodiments include methods wherein said activated mesenchymal stem cells is stimulated by exposure to a cytokine.

Preferred embodiments include methods wherein said cytokine activates the JAK/STAT pathway.

Preferred embodiments include methods wherein said cytokine is interleukin-1 beta

Preferred embodiments include methods wherein said cytokine is interferon alpha.

Preferred embodiments include methods wherein said cytokine is interferon beta.

Preferred embodiments include methods wherein said cytokine is interferon tau.

Preferred embodiments include methods wherein said cytokine is interleukin-1 beta.

Preferred embodiments include methods wherein said cytokine is tumor necrosis factor alpha.

Preferred embodiments include methods wherein said cytokine is tumor necrosis factor beta.

Preferred embodiments include methods wherein said cytokine is interleukin-6.

Preferred embodiments include methods wherein said cytokine is interleukin-8.

Preferred embodiments include methods wherein said cytokine is interleukin-9.

Preferred embodiments include methods wherein said cytokine is interleukin-11.

Preferred embodiments include methods wherein said cytokine is interleukin-17.

Preferred embodiments include methods wherein said cytokine is interleukin-18.

Preferred embodiments include methods wherein said cytokine is interleukin-21.

Preferred embodiments include methods wherein said cytokine is interleukin-23.

Preferred embodiments include methods wherein said cytokine is interleukin-27.

Preferred embodiments include methods wherein said cytokine is interleukin-33.

Preferred embodiments include methods wherein said T cells express CD39.

Preferred embodiments include methods wherein said T cells express IL-10.

Preferred embodiments include methods wherein said T cells express leukemia inhibitory factor.

Preferred embodiments include methods where in the potency of the conditioned/enhanced media is measured by HGF levels.

Preferred embodiments include methods where the T cell will also decrease rejection episodes in pancreatic islet transplantation (autologous/allogeneic/xenogeneic).

Preferred embodiments include methods where the T cell will decrease rejection episodes in solid tissue transplantation.

Preferred embodiments include methods where the T cell will decrease rejection episodes in solid organ transplantation.

An augmented T regenerative cell created by utilizing the supernatant of a stimulated mesenchymal stem cell in the culture process.

The composition of claim 271, where the mesenchymal stem cell is autologous.

The composition of claim 271, where the mesenchymal stem cell is allogeneic.

The composition of claim 271, where the mesenchymal stem cell is xenogeneic.

The composition of claim 271, where the mesenchymal stem cell is derived from an induced pluripotent stem cell.

The composition of claim 271, where the stimulant is conconavalin-A, phytohemagluttinin, pokeweed mitogen, Galanthus nivalis Lectin, or Cytokine interleukin-1 beta, interleukin-2, interleukin-3, interleukin-4, interleukin-7, interleukin-10, interleukin-12, interleukin-18, interleukin-17, interleukin-20, interleukin-35, interferon alpha, interferon beta, interferon gamma, interferon omega.

The composition of claim 271, where the regenerative T cell expresses at least 5 of the following CD4, CD39, FOXP3, CD25, CD34, CD45, CD73, CD3, CD127, CTLA-4, GITR, LAG-3, LRRC32 and Neuropilin-1

The composition of claim 271, where the supernatant results in an increase in regenerative T cell production of at least 5-10%.

The composition of claim 271, where the supernatant results in an increase in regenerative T cell production of at least 15-25%.

The composition of claim 271, where the supernatant results in an increase in regenerative T cell production of at least 26-35%.

The composition of claim 271, where the supernatant results in an increase in regenerative T cell production of at least 36-45%.

The composition of claim 271, where the supernatant results in an increase in regenerative T cell production of at least 46-55%.

The composition of claim 271, where the supernatant results in an increase in regenerative T cell production of at least 56% or greater.

An augmented T regenerative cell secreting IL-35 created by utilizing the supernatant of a stimulated mesenchymal stem cell in the culture process.

The composition of claim 284, where the mesenchymal stem cell is autologous.

The composition of claim 284, where the mesenchymal stem cell is allogeneic.

The composition of claim 284, where the mesenchymal stem cell is xenogeneic.

The composition of claim 284, where the mesenchymal stem cell is derived from an induced pluripotent stem cell.

The composition of claim 284, where the stimulant is conconavalin-A, phytohemagluttinin, pokeweed mitogen, Galanthus nivalis Lectin, or Cytokine interleukin-1 beta, interleukin-2, interleukin-3, interleukin-4, interleukin-7, interleukin-10, interleukin-12, interleukin-18, interleukin-17, interleukin-20, interleukin-35, interferon alpha, interferon beta, interferon gamma, interferon omega.

The composition of claim 284, where the regenerative T cell expresses at least 5 of the following CD4, CD39, FOXP3, CD25, CD34, CD45, CD73, CD3, CD127, CTLA-4, GITR, LAG-3, LRRC32 and Neuropilin-1

The composition of claim 284, where the supernatant results in an increase in regenerative T cell production of at least 5-10%.

The composition of claim 284, where the supernatant results in an increase in regenerative T cell production of at least 15-25%.

The composition of claim 284, where the supernatant results in an increase in regenerative T cell production of at least 26-35%.

The composition of claim 284, where the supernatant results in an increase in regenerative T cell production of at least 36-45%.

The composition of claim 284, where the supernatant results in an increase in regenerative T cell production of at least 46-55%.

The composition of claim 284, where the supernatant results in an increase in regenerative T cell production of at least 56% or greater.

### DETAILED DESCRIPTION OF THE INVENTION

The invention provides means of enhancing pancreatic islet survival through modification of the hepatic microenvironment prior to administration of said cells. In some embodiments the invention provides the administration of tolerogenic cells, followed by T and/or Treg cells that have been conditioned by regenerative cells, followed by administration of the pancreatic islets. This may also occur in the reverse order to decrease rejection episodes.

As used herein, the term “MHC class I” or “MHC I” refers to the human Major Histocompatibility Complex class I molecules, binding peptides or genes. The human MHC region, also referred to as HLA, is found on chromosome six and includes the class I region and the class II region. Within the MHC class I region are found the HLA-A, HLA-B or HLA-C subregions for class I 0: chain genes. The human gene for β2-microglobulin is located outside the MEW complex on a separate chromosome. As used herein, the term “MHC class I molecule” means a complex of an MEW class I a chain and a β2-microglobulin chain. MHC class I molecules normally bind peptides which are generated in the cytosol and transported to the endoplasmic reticulum. After binding these peptides, the class I MHC-peptide complex is presented on the cell surface where it may be recognized by T cells. The majority of bound peptides have a length of 8-10 amino acids, although they may be as long 16 or as short as 2 (Udaka et al., (1993) Proc. Natl. Acad. of Sci. (USAt 90:11272-11276). See, generally, Roitt et al., eds. Immunology (1989) Gower Medical Publishing, London.

As used herein, the term “MHC class II” or “MHC II” refers to the human Major Histocompatibility Complex class II molecules, binding peptides or genes. The human MHC region, also referred to as HLA, is found on chromosome six and includes the class I region and the class II region. Within the MHC class II region are found the DP, DQ and DR subregions for class II cc chain and 0 chain genes (i.e., DPa, DPP, DQa, DQP, DRa, and DRβ). As used herein, the term “MHC class II molecule” means a complex of an MHC class II α chain and an MHC class II β chain. MHC class II molecules normally bind peptides in an intracellular processing compartment and present these peptides on the surface of antigen presenting cells to T cells. The majority of bound peptides have a length of 13-18 amino acids but it is the peptide side chains of an approximately 9 amino acid core segment that occupy pockets of the MHC class II binding cleft and determine the specificity of binding (Brown et al., (1993) Nature 364:33-39; Stem et al., (1994) Nature 368:215-221). See, generally, Roitt et al., eds. Immunology (1989) Gower Medical Publishing, London.

The term “oligonucleotide” refers to a short polymer of nucleotides and/or nucleotide analogs. The term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phophoroamidate, and/or phosphorothioate linkages. Preferred RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

As used herein, the “5′ end”, as in the 5′ end of an antisense strand, refers to the 5′ terminal nucleotides, e.g., between one and about 5 nucleotides at the 5′ terminus of the antisense strand. As used herein, the “3′ end”, as in the 3′ end of a sense strand, refers to the region, e.g., a region of between one and about 5 nucleotides, that is complementary to the nucleotides of the 5′ end of the complementary antisense strand.

As used herein, “modulation” may constitute any alteration at any point in time of the relative activity or abundance of, for example, a gene, gene product, or pathway, as compared to wild-type levels. (Examples of such modulation include: gene knockouts, transgenic expression of a gene or mutant form of a gene, expression of a mutant form of a native gene, underexpression and overexpression of a gene.) An “RNAi modulatory compound” is therefore any compound capable of modulation in any manner of RNAi.

As used herein, a “reduced activity” is one that is at least 5% less than that of the polypeptide or protein encoded by the wild-type nucleic acid molecule or gene, preferably at least 5-10% less, more preferably at least 10-25% less and even more preferably at least 25-50%, 50-75% or 75-100% less than that of the polypeptide or protein encoded by the wild-type nucleic acid molecule or gene. Ranges intermediate to the above-recited values, e.g., 75-85%, 85-90%, 90-95%, are also intended to be encompassed by the present invention. As used herein, a “reduced activity” also includes an activity that has been deleted or “knocked out” (e.g., approximately 100% less activity than that of the polypeptide or protein encoded by the wild-type nucleic acid molecule or gene). Likewise, an “enhanced activity” is one that is at least 5% greater than that of the polypeptide or protein encoded by the wild-type nucleic acid molecule or gene, preferably at least 5-10% greater, more preferably at least 10-25% greater and even more preferably at least 25-50%, 50-75% or 75-100% greater or 100% or more greater (two-fold or greater elevated) than that of the polypeptide or protein encoded by the wild-type nucleic acid molecule or gene. Ranges intermediate to the above-recited values, e.g., 75-85%, 85-90%, 90-95%, are also intended to be encompassed by the present invention.

Activity can be determined according to any well accepted assay for measuring activity of a particular protein of interest. Activity can be measured or assayed directly, for example, by measuring an activity of a protein isolated or purified from a cell. Alternatively, an activity can be measured or assayed within a cell or in an extracellular medium or in a crude extract of cells. Additionally, activity of a targeted protein may also be measured in a whole organism.

The term “tissue”, as used herein, refers to any biological entity derived from an organism (directly or via an isolated progenitor cell or population) that is comprised of cells, including whole organs, organ sections and subsections, tumor cells, cells, cell lines, etc. The term also includes plant cells, as used herein.

The term “perfusion”, as used herein, refers to the act of pouring over or through, especially the passage of a fluid through the vessels of a specific organ. In specific embodiments of the instant invention, fluids containing RNAi agents are perfused through the vasculature of transplant tissues.

The terms “apoptosis” or “programmed cell death,” refers to the physiological process by which unwanted or useless cells are eliminated during development and other normal biological processes. Apoptosis, is a mode of cell death that occurs under normal physiological conditions and the cell is an active participant in its own demise (“cellular suicide”). It is most often found during normal cell turnover and tissue homeostasis, embryogenesis, induction and maintenance of immune tolerance, development of the nervous system and endocrine-dependent tissue atrophy. Apoptosis may also be triggered by external events and stimuli, such as ischemic injury in the case of certain preferred embodiments of the instant invention. Cells undergoing apoptosis show characteristic morphological and biochemical features. These features include chromatin aggregation, nuclear and cytoplasmic condensation, partition of cytoplasm and nucleus into membrane-bound vesicles (apoptotic bodies) which contain ribosomes, morphologically intact mitochondria and nuclear material. In vivo, these apoptotic bodies are rapidly recognized and phagocytized by either macrophages or adjacent epithelial cells. Due to this efficient mechanism for the removal of apoptotic cells in vivo no inflammatory response is elicited. In vitro, the apoptotic bodies as well as the remaining cell fragments ultimately swell and finally lyse. This terminal phase of in vitro cell death has been termed “secondary necrosis.”

“Inhibition of gene expression” refers to the absence (or observable decrease) in the level of protein and/or mRNA product from a target gene. “Specificity” refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

The term “glucose-activated insulin response”, as used herein, refers to the process by which the islet cells of the pancreas sense and respond to glucose levels (e.g., circulating blood glucose levels for islet cells in vivo). High circulating blood glucose levels will typically trigger elevated production and secretion of insulin by pancreatic islet cells in vivo, through molecular signaling mechanisms that are widely recognized in the art. These glucose signaling responses may be modulated by RNAi agents (e.g., shRNA, miRNA, siRNA, etc.; Katayama, K., et al. FEBS Lett. 2004 560, 178-82; Huang, A., et al FEBS Lett. 2004 558, 69-73).

In one embodiment the invention provides means of enhance cross-presentation in an allogeneic manner in the hepatic microenvironment. One means of enhancing tolerogenicity of allogeneic cross-presentation is through providing interleukin-10 into the hepatic microenvironment prior to, concurrently with, and/or subsequently to administration of pancreatic islets. In some embodiment dendritic cells are administered in an immature state as a means of inducing the production of IL-10 locally. Generation of immature dendritic cells can be performed by culture in agents that inhibit NF-kappa B activation such as hepatic growth factor.

Generation of pancreatic islets for the purpose of the invention may be performed by numerous means known in the field. In one embodiment, a protocol similar to the one below or identical to it is followed:
1. Step for swelling pancreas with enzyme (Swelling Step). Firstly, a wire net with a tray underneath are placed in a clean bench. Next, the pancreas is placed on the wire net, into which an enzyme is introduced from a vein of the pancreas by using a catheter. As for the enzyme, it is an enzyme formed by blending collagenase I and collagenase II with neutral protease having a high specific activity (by Roche Diagnostics K.K., product name: Liberase). After a period of time, the enzyme oozes from the pancreas to the outside, and is drawn up manually and sprinkled to the pancreas again. By repeating these operations for about 25 minutes, the pancreas is swelled with the enzyme. In order to prevent any enzymatic reaction, the enzyme is chilled to about 4° C.
2. Step for degrading pancreas with enzyme (Degradation Step). A circuit for carrying out the enzyme reaction and mechanical degradation provided with a stainless container and a temperature-controlling means was subjected to priming with the Hanks' buffer salt solution (HBSS). In the stainless container, the pancreas segments having been cut into pieces by a surgical knife and six iron balls having a marble size (outer diameter: about 10 mm, weight: about 20 g) were put. The temperature of the temperature-controlling means is set to about 40° C., and the HB SS was circulated at a liquid feed velocity of about 150 ml/min to initiate the enzyme reaction. At the same time, by placing a stainless container housing to a vibratory apparatus to initiate mechanical degradation with the amplitude of about 200 mm and a cycle of about 60 times/sec. In 60 minutes, the iron balls in the stainless container were removed and the liquid material was collected.
3. Step for Separating Pancreatic Islet from Other Tissues by Density Gradient Centrifugation Method (Separation Step). The liquid material obtained in the above (2) was treated by a density gradient centrifugation method to separate pancreatic islet from the liquid material. For the density gradient centrifugation apparatus, one that had been purchased from COBE Inc. (trade name: COBE2991) was used. For the specific gravity liquid for use in the centrifugation, a Bicoll liquid was selected. The centrifugation was carried out under the condition of rotation number of about 2300 rpm. Firstly, a liquid having a specific density of 1.077 was charged in the centrifugal circuit, into which a liquid having a specific density of 1.100 was gradually flowed while being subjected to centrifugation, resulting in a density gradient of 1.077-1.100. After that, the liquid material obtained in the degradation step was flowed into the circuit, and the eluted liquid was sequentially fractionated from the collection port into polystyrene T-type 25 cm2 flasks in increments of about 20 ml and observed with a microscope. Then, it was decided that a large amount of pancreatic islet was contained in the fraction that was obtained after the liquid of about 200 ml had been eluted after the start of the centrifugation, and so about 100 ml of the eluate after that was collected. The collected eluate was put in a 250 ml centrifugal tube, which was then subjected to centrifugation at 224 g to remove the specific gravity liquid. After that, the pancreatic islet was suspended in a CMRL medium again.

Another more detailed means to obtain pancreatic islets is described to assist the practitioner in the embodiment of the invention. The methods of isolating pancreatic islet cells for transplantation are provided and incorporated by reference. In one embodiment, the methodology described in US patent application #20060182722 are provided: Pancreases can be obtained from male or female donors in accordance with federal regulations (e.g., 21 C.F.R. .sctn.1270) and techniques developed for combined liver and pancreaticoduodenal procurement (Marsh et al., Surg. Gynecol. Obstet. 1989; 168:254-258). Donors typically range in age from 15 to 50 years old. General exclusion criteria include, for example, systemic bacterial infections, viruses such as human immunodeficiency virus (HIV), human T-cell lymphotrophic virus (HTLV), hepatitis B virus, or hepatitis C virus (HCV), a history of diabetes, extracranial tumors, and risk factors for AIDS. Donor pancreases can be preserved using the two-layer pancreas preservation method, which improves pancreatic tissue adenosine triphosphate (ATP) content, increases the yield of islets isolated from a stored pancreas, allows use of marginal donor pancreases for islet isolation and transplantation, improves the islet isolation success rate, and preserves the integrity of the isolated islets (e.g., such that isolated islets can reverse diabetes). In general, cold University of Wisconsin (UW) Solution (ViaSpan®, DuPont Pharma, Wilmington, Del.) (see U.S. Pat. Nos. 4,798,824 and 4,879,283) or modified UW solution can be poured on top of an equal volume of cold perfluorodecalin (FluoroMed, L. P., Round Rock, Tex.).

Typically, the two-layer preservation method is performed in an organ shipping container, which has, for example, a removable lid with a stainless steel mesh plate attached thereto, and inlet and outlet ports. See, for example, the organ shipping container of U.S. Pat. No. 6,490,880. Two layers are formed after adding ViaSpan or modified-UW solution to the perfluorodecalin as the specific gravity of perfluorodecalin is greater than ViaSpan® and modified-UW solution. Modified UW solution includes 0.35 to 0.45 g/L potassium hydroxide, 3.00 to 4.00 g/L monosodium phosphate monohydrate, 0.05 to 1.00 g/L calcium chloride dihydrate, 1.10 to 1.30 g/L magnesium sulfate heptahydrate, 33.00 to 38.00 g/L lactobionic acid, 4.00 to 5.00 g/L L-histidine, 15.00 to 20.00 g/L raffinose, 4.00 to 5.00 g/L sodium hydroxide, 15.00 to 25.00 g/L penta starch, 1.00 to 1.50 g/L adenosine, and 0.75 to 1.50 g/L glutathione. In particular, the modified UW solution can include 0.39 g/L potassium hydroxide, 3.45 g/L monosodium phosphate monohydrate, 0.074 g/L calcium chloride dihydrate, 1.23 g/L magnesium sulfate heptahydrate, 35.83 g/L lactobionic acid, 4.66 g/L L-histidine, 17.84 g/L raffinose, 4.60 g/L sodium hydroxide, 20.00 g/L penta starch, 1.34 g/L adenosine, and 0.92 g/L glutathione. Typically, the perfluorodecalin is oxygenated for 30-70 minutes (e.g., 40-60 minutes). For example, medical grade oxygen can be filtered through a 0.2 mm filter (Gelman Sciences, Ann Arbor, Mich.) and the inlet port of the shipping container at a rate of 2.5 L/min. Preferably, the cold storage time of the donor pancreas is less than 12 hours (e.g., less than 10, 8, 6, 4, or 2 hours). Upon receipt of a donor pancreas, integrity of the shipping container can be verified by visual inspection. The pancreas can be removed and rinsed with cold transport solution containing 8.00 to 10.00 g/L mannitol, 3.00 to 6.00 g/L L-histidine, 18.00 to 21.00 g/L gluconic acid, 0.50 to 2.00 g/L potassium hydroxide, 0.01 to 0.05 g/L calcium chloride, 0.50 to 2.00 g/L magnesium sulfate, 0.40 to 0.80 g/L nicotinamide, 0.30 to 0.70 g/L pyruvate, and 1.50 to 3.50 g/L potassium phosphate monobasic. For example cold transport solution can include 8.50 to 9.50 g/L (e.g., 9.11 g/L) D-mannitol, 4.00 to 5.00 g/L (e.g., 4.67 g/L) L-histidine, 18.50 to 20.50 g/L (e.g., 19.63 g/L) D-gluconic acid sodium salt, 0.80 to 1.40 g/L (e.g., 1.12 g/L) potassium hydroxide, 0.025 to 0.045 g/L (e.g., 0.037 g/L) calcium chloride dihydrate, 1.00 to 1.50 g/L (e.g., 1.23 g/L) magnesium sulfate heptahydrate, 0.55 to 0.65 g/L (e.g., 0.61 g/L) nicotinamide, 0.50 to 0.60 g/L (e.g., 0.55 g/L) sodium pyruvate, and 2.50 to 3.25 g/L (e.g., 2.72 g/L) potassium phosphate monobasic. Islets can be isolated from the donor pancreas using an automated method of pancreatic tissue dissociation. See, for example, Ricordi et al., Diabetes 1988; 37:413-420. This method includes the general steps of: 1) dissection; 2) distension; 3) dissociation; and 4) collection. Dissection of the pancreas can include removing extraneous fat (while retaining some fat to minimize leaking during distension), and non-pancreatic tissue. Typically, about 80% to about 95% of the fat is removed. The dissected pancreas can be incubated in a topical antibiotic solution containing, for example, gentamicin (Elkins-Sinn, Inc.), Cefazolin (SmithKline Beecham Pharmaceutical), and amphotericin-B (Apothecon®) in cold transport solution, then can be serially rinsed in phenol red-free Hanks' Balanced Salt Solution (Mediatech, Inc., Herndon, Va.). The pancreas can be divided at the neck into the ‘body and tail’ and ‘head’ and the following steps performed on each part. In general, the pancreatic duct can be cannulated with an angiocatheter (16-20 gauge) and the pancreas perfused under controlled conditions, including an initial pressure of 80 mmHg followed by an increase in pressure to 180 mmHg for the remainder of the distension procedure. Phase I solution can be used to perfuse the pancreas. Phase I solution includes 5.00 to 6.00 g/L mannitol, 0.50 to 0.70 g/L sodium hydroxide, 5.00 to 7.00 g/L sodium chloride, 0.25 to 0.40 g/L potassium hydroxide, 0.05 to 0.15 g/L calcium chloride, 0.15 to 0.25 g/L magnesium sulfate, and 3.00 to 4.00 g/L sodium phosphate monobasic. For example, Phase I solution can include 5.47 g/L D-mannitol, 0.60 g/L sodium hydroxide, 6.14 g/L sodium chloride, 0.33 g/L potassium hydroxide, 0.11 g/L calcium chloride dihydrate, 0.20 g/L magnesium sulfate heptahydrate, and 3.45 g/L sodium phosphate monobasic. Typically, the Phase I solution contains 1,000 to 3,600 Wunsch units (collagenase activity) or 28,000 to 128,500 caseinase units (proteolytic activity) of collagenase. For example, the Phase I solution can include 1500 to 3000 (e.g., 1,562 to 2,954 or 2,082 to 2,363) Wunsch units, or 42,000 to 108,000 (e.g., 42,328 to 107,064 or 56,437 to 85,651) caseinase units of collagenase. A suitable collagenase includes Liberase™HI (Roche Molecular Biochemicals, Indianapolis, Ind.), which has been specifically formulated for human islet isolation procedures. See, Linetsky et al., Diabetes 1997; 46:1120-1123. Preferably, powdered Liberase™HI is reconstituted at least 20 minutes before, but less than 2 hours before, addition to the Phase I solution. The Phase I solution also can include a protease inhibitor (e.g., a trypsin inhibitor such as 4-(2-aminoethyl)-benzenesulfonyl fluoride hydrochloride (Pefabloc® SC PLUS), TLCK (1-Chloro-3-tosylamido-7-amino-2-heptanone HCl), or trypsin inhibitor from soybean). For example, the Phase I solution can include 0.05 to 0.15 mg/mL of Pefabloc® SC PLUS, which specifically inhibits endogenous proteases and decreases auto-digestion. The Phase I solution also can include 8 to 12 units/mL of heparin (e.g., Monoparin®, Accurate Chemical and Scientific Corporation). For example, the Phase I solution can include 10 units/mL of heparin.

In some embodiments, the Phase I solution contains 1,000 to 3,600 Wunsch units of collagenase, 0.05 to 0.15 mg/mL of a trypsin inhibitor, and 10 units/mL of heparin. After a sufficient period of time of cold perfusion, e.g., 8-20 minutes, the distended pancreas can be further trimmed of remaining capsule and placed into a dissociation chamber (e.g., a sterile stainless steel chamber (Wahoff et al., Ann. Surg. 1995; 222:562-579), also known as a Ricordi chamber). Collagenase that “leaked” from the distended pancreas can be added to the chamber. Typically, the Ricordi chamber is in a circulation system that includes a heat exchange coil (e.g., a stainless steel coil), a pump, a temperature monitor and sensor, a loading flask, a fluid collection flask, a sample collecting flask, and tubes for fluidly connecting components. Flow direction can be controlled using, for example, valves or clamps. The heat exchange coil can be placed in a water bath. One embodiment of a circulation system that contains a Ricordi chamber, a stainless steel coil for heat exchange, six tubes with small diameter (Master Flex tubing, size 16), four tubes with large diameter (Master Flex tubing, size 17), steel 3-way stopcock for sampling, four plastic clamps, 250 mL conical tube, tri-pour graduated disposable beaker, 1000 mL, bell-shaped plastic cover, two T-connectors, one T-connector with luer lock port, and one Y-connector, 18 inch steel ring stand with two arms, Ismatec pump, Mon-a-therm temperature monitor and sensor, and water bath. The system can be filled with Phase I solution and air evacuated to begin the digestion phase. In particular, Phase I solution can be allowed to flow from the loading flask through the pump, heat exchange coil, and Ricordi chamber to the fluid collecting flask. After 10% to 30% of the volume of Phase I solution reaches the fluid collecting flask, the flow of the system can be adjusted such that the Phase I solution is recirculated through the system, i.e., the Phase I solution flows from the fluid collecting flask to the chamber and from the chamber to the fluid collecting flask. The chamber can be agitated while the fluid is being recirculated to aid tissue dissociation. Temperature of the fluid can be maintained at 25° C. to 37° C. The collection phase can begin once there is an increase in the amount of tissue liberated from the chamber, most or all of the islets are free of the surrounding acinar tissue, intact islets are observed, and the acinar tissue becomes finer (small cell clusters). Diphenylthiocarbazone (DTZ) staining can be used to distinguish islets from non-islet tissue. See, Latif et al., Transplantation 1988; 45:827-830. DTZ selectively binds to the zinc-insulin complex in islet beta cell granules, and results in a red staining of the islets. DTZ staining provides a rapid means for discrimination of islet from acinar tissue, and the positive reaction indicates that insulin-containing beta cells are present.

During the collection phase, the temperature of the system can be reduced to about 10° C. to about 30° C. Fluid in the fluid collecting flask can be allowed to flow through the pump and heat exchange coil into the Ricordi chamber, and Phase II solution (RPMI 1640, catalog #99-595-CM, Mediatech, Inc., Herndon, Va.) can be added to a loading flask. The Phase II solution can be pumped through the circulation system to dilute the collagenase and to wash the tissue. Digested material can be collected in flasks containing Phase II solution and human serum albumin (HSA), and the collected material washed two to five times using cold storage solution. Cold storage solution can include 16.00 to 20.00 g/L raffinose, 4.00 to 6.00 g/L histidine, 4.00 to 5.00 g/L sodium hydroxide, 30.00 to 40.00 g/L lactobionic acid, 0.30 to 0.50 g/L potassium hydroxide, 0.05 to 0.10 g/L calcium chloride, 1.00 to 1.50 g/L magnesium sulfate, 3.00 to 4.00 g/L sodium phosphate monobasic, 19.00 to 21.00 g/L pentastarch, 8.00 to 12.00 U/mL heparin, and 8.00 to 12.00.mu·g/mL insulin. For example, cold storage solution can include 17.84 g/L D (+) raffinose, 4.66 g/L L-histidine, 4.60 g/L sodium hydroxide, 35.83 g/L lactobionic acid, 0.39 g/L potassium hydroxide, 0.39 g/L calcium chloride dihydrate, 1.23 g/L magnesium sulfate heptahydrate, 3.45 g/L sodium phosphate monobasic, 2% penta starch, 10 U/mL heparin, and 10.mu·g/mL insulin. Cold storage solution can be made by combining H-Phase II solution (80% by volume) with 10% penta starch (i.e., 100 g/L) (20% by volume), and adding 8.00 to 12.00 U/mL heparin, and 8.00 to 12.00.mu·g/mL insulin. H-Phase II solution can include 16.00 to 20.00 g/L raffinose, 4.00 to 6.00 g/L histidine, 4.00 to 5.00 g/L sodium hydroxide, 30.00 to 40.00 g/L lactobionic acid, 0.30 to 0.50 g/L potassium hydroxide, 0.05 to 0.10 g/L calcium chloride, 1.00 to 1.50 g/L magnesium sulfate, and 3.00 to 4.00 g/L sodium phosphate monobasic. The pH of H-Phase II solution can be adjusted to a pH of 7.3-7.5 using hydrochloric acid or sodium hydroxide. Density of H-Phase II solution typically is 1.063.+−0.0.003. For example, H-Phase II solution can include 17.84 g/L D (+) raffinose, 4.66 g/L L-histidine, 4.60 g/L sodium hydroxide, 35.83 g/L lactobionic acid, 0.39 g/L potassium hydroxide, 0.39 g/L calcium chloride dihydrate, 1.23 g/L magnesium sulfate heptahydrate, and 3.45 g/L sodium phosphate monobasic. The washed tissue can be resuspended in capping layer solution and HSA (e.g., 25% HSA). Capping layer solution can include 16.00 to 20.00 g/L raffinose; 4.00 to 6.00 g/L histidine; 4.00 to 5.00 g/L sodium hydroxide; 30.00 to 40.00 g/L lactobionic acid; 0.30 to 0.50 g/L potassium hydroxide; 0.05 to 0.10 g/L calcium chloride; 1.00 to 1.50 g/L magnesium sulfate; 3.00 to 4.00 g/L sodium phosphate monobasic; and 19.00 to 21.00 g/L pentastarch. For example, capping layer solution can have a density of 1.035 to 1.036 g/cm·sup·3 and can include 17.84 g/L D (+) raffinose, 4.67 g/L L-Histidine, 4.6 g/L sodium hydroxide, 35.83 g/L lactobionic acid, 0.393 g/L potassium hydroxide, 0.07 g/L calcium chloride dihydrate, 1.23 g/L magnesium sulfate heptahydrate, 3.45 g/L sodium phosphate monobasic, and 2% penta starch. Capping layer solution can be made by combining H-Phase II solution (80% by volume) with 10% penta starch (i.e., 100 g/L) (20% by volume). Islets can be purified using continuous density gradient separation. Gradients can be prepared using iodixanol (OptiPrep™, Nycomed, Roskilde, Denmark) (density 1.32 g/cm·sup·3) and capping layer solution, cold storage solution, and/or high-density (HD) stock solution. HD stock solution can include 16.00 to 20.00 g/L raffinose; 4.00 to 6.00 g/L histidine; 4.00 to 5.00 g/L sodium hydroxide; 30.00 to 40.00 g/L lactobionic acid; 0.30 to 0.50 g/L potassium hydroxide; 0.05 to 0.10 g/L calcium chloride; 1.00 to 1.50 g/L magnesium sulfate; 3.00 to 4.00 g/L sodium phosphate monobasic; 15.00 to 25.00 g/L pentastarch; and 200 to 300 ml/L iodixanol. The density of the HD stock solution typically is 1.112.+−0.0.003 g/cm·sup·3. For example, HD stock solution can include 17.84 g/L D (+) raffinose, 4.67 g/L L-Histidine, 4.6 g/L sodium hydroxide, 35.83 g/L lactobionic acid, 0.39 g/L potassium hydroxide, 0.07 g/L calcium chloride dihydrate, 1.23 g/L magnesium sulfate heptahydrate, 3.45 g/L sodium phosphate monobasic, 20 g/L penta starch, and 250 mL/L iodixanol (Optiprep™). In some embodiments, HD stock solution also can include 8.00 to 12.00 U/mL of heparin and/or 8.00 to 12.00.mu·g/mL insulin. A bottom density gradient solution having a density that ranges from 1.08 to 1.13 g/cm·sup·3 can be prepared by mixing HD stock solution and cold storage solution. A light density gradient solution having a density of 1.050 to 1.080 g/cm·sup·3 can be made by mixing iodixanol and cold storage solution, while a heavy density gradient solution having a density of 1.06 to 1.13 g/cm·sup·3 can be made by mixing cold storage solution and HD stock solution.

A continuous gradient can be made, for example, in a dual chamber gradient maker, by combining the light and heavy density gradient solutions. The bottom density gradient can be transferred to a cell processing bag for a cell separator such as the Cobe 2991 cell separator (Lakewood, Colo.), and the continuous gradient can be overlaid on the bottom density gradient. The resuspended tissue (as described above) can be placed on the continuous gradient followed by a capping layer solution then the gradient can be spun to separate the islets. Fractions can be collected and assayed for the presence of islets as described below. Fractions with islet purities (percentage of DTZ positive cells)>10% can be combined for culture. Purified islets can be cultured using a chemically defined culture medium that is effective for maintaining viability of human pancreatic islets under culture conditions. Typically, islets are cultured at a temperature of 22° C. to 37° C. and in an atmosphere of 95% air and 5% CO₂. In some embodiments, islets can be cultured in an atmosphere of room air. Viability of islets can be assessed using trypan blue or a fluorescent dye inclusion/exclusion assay. See, for example, Barnett et al., Cell Transplant. 2004; 13(5):481-8. The chemically defined culture medium can include one or more of the following: insulin, zinc sulfate, selenium, transferrin, sodium pyruvate, HEPES (N-[2-Hydroxyethyl]piperazine-N[2-ethanesulfonic acid]), HSA, and heparin. For example, the chemically defined culture medium can include 5.50 to 7.50.mu·g/mL insulin, 15 to 18.mu·M zinc sulfate, 5.50 to 7.50 ng/mL selenium (e.g., selenous acid), and 5.50 to 7.50.mu·g/mL transferrin (e.g., human transferrin). Such a culture medium further can include one or more of the following: 3 to 7 mM sodium pyruvate, 20 to 30 mM HEPES, 0.50 to 1.50 mg/mL HSA, 8.00 to 12.00 U/mL of heparin, 1 to 3 mM L-Alynyl-L-glutamine, and 4.50 to 6.50.mu·g/mL linoleic acid. Typically, when the cells are to be cultured under 95% room air and 5% CO₂, the chemically defined culture medium includes bicarbonate (e.g., 1.75 to 2.75 g/L such as 2.2 g/L). The bicarbonate concentration can be reduced if the cells are cultured in 100% room air. In some embodiments, the chemically defined culture medium also includes an antibiotic such as ciprofloxacin (Bayer Corporation). In one embodiment, a chemically defined culture medium can be CMRL 1066 (Mediatech, Inc., Herndon, Va.) supplemented with 25 mM HEPES, 2 mM L-Alynyl-L-Glutamine, 5 mM sodium pyruvate, 1% (vol/vol), ITS additive (6.25.mu·gg/mL human recombinant insulin, 6.25.mu·g/mL human transferrin, 6.25 ng/mL selenous acid, 1.25 mg/mL HSA, 5.35.mu·g/mL linoleic acid), 16.7.mu·M zinc sulfate, 20.mu·g/mL ciprofloxacin (Bayer Corporation) and 0.5% final concentration of 25% HSA. Human Insulin-like Growth Factor-I (IGF-I, GRO PEP Pty Ltd, Adelaide, South Australia) can be added to the islet culture. For example, 90 to 110 ng/mL (e.g., 100 ng/mL) of IGF-1 can be added to the culture. Typically, the islets are cultured overnight at 37° C., then for an additional 1 to 3 days at 22° C. Pretransplant culture of islets can provide beneficial metabolic and immunologic effects. For example, culturing islets for two days can improve the metabolic efficacy of the cultured islets relative to freshly isolated islets. Pretransplant islet culture also can allow time for T-cell-directed immunosuppression to be achieved in the recipient before the transplant. Without being bound to a particular mechanism, achieving T-cell-directed immunosuppression may reduce islet-directed immune responses mediated by autoreactive, primed T cells to which the transplanted islets are immediately exposed. As described herein, delaying transplantation until two days after the initiation of therapy with T-cell-depleting antibodies prevents exposure of transplanted islets to the cytokine release associated, to varying degrees, with the first and second antibody infusions. Furthermore, pretransplant culture of islets allows quality control studies to be performed before the infusion of tissue.

Purified islet cells can be cryopreserved by suspending the cells in a cryopreservative such as dimethylsulfoxide (DMSO) or ethylene glycol, or a mixture of cryopreservatives. See, for example, Miyamoto et al., Cell Transplant 2001; 10(4-5):363-71; Evans et al., Transplantation 1990; 50(2):202-206; and Lakey et al., Cell Transplant 1996; 5(3):395-404. Islet cells can be cryopreserved after purification or culture. Typically, the cryopreservative is added in a stepwise fashion and the islets are slow cooled to −40° C. then stored at −196° C. Islets can be rapidly thawed (e.g., in a 37° C. water bath) and assayed before use. Cryopreservation can allow for long-term storage of these cells for later transplantation or other purpose. Cryopreserving collections of purified populations of islets cells is particularly useful for producing an islet bank.

Preparations of isogenic islet cells purified using the methods described herein typically result in successful transplants in at least 55% (e.g., at least 60%, 65%, 70%, 75%, 80%, 85%, 90%, or 95%) of the patients. A transplant is considered a success when a patient sustains insulin independence, normoglycemia, and freedom from hypoglycemia for at least one year after a single-donor islet transplant.

Preparations of purified islet cells can be assayed to confirm that the islets have sufficient potency to be transplanted. As used herein, “transplant potency” refers to an estimate of the probability that the preparation of islets can be successfully transplanted in a patient and is based on one or more of the following parameters: safety of the islet preparation, islet cell number, cellular composition of islet preparation, number of beta cells, insulin content, tissue volume, viability, ATP content, percent of islet equivalents recovered after cell culture, percent necrotic and apoptotic cells, glucose-stimulated insulin release, and oxygen consumption rate (OCR). For example, transplant potency can be estimated based on the ATP/DNA ratio, OCR/DNA ratio, and beta cell number. Preparations of purified islets that have at least a 60% probability of constituting a successful transplant are particularly useful. Safety of an islet preparation can be determined by assaying for the presence of aerobic and anaerobic organisms and fungi, mycoplasma, and other adventitious agents (e.g., viruses) using known techniques. For example, a sample can be Gram stained to detect bacteria. Islet cells suitable for transplantation do not contain detectable organisms and are functionally sterile. Assessing safety also can include measuring endotoxin present in the preparation. Islet cell preparations suitable for transplant have an endotoxin content of 1.7 EU/mL (5 EU/kg recipient body weight) or less. Islet cell number can be assessed by staining with DTZ and quantifying the size distribution of the stained cells using a light microscope with ocular micrometer. See, Ricordi et al., Acta Diabetol. Lat. 1990; 27:185-195. Islet volume can be calculated, based on the assumption that islets are spherical, and the number of islets is expressed in terms of islet equivalents (IE), with one IE equal to a 150.mu·m diameter islet. Preparations of islets containing at least 2.2×10⁵ IE (e.g., 2.7×10⁵, 3.5×10⁵, 4.5×10⁵, 5.5×10⁵, 7.0×10⁵, 9.0×10×⁵, 1.110⁶, or 1.4×10⁶ IE) are particularly useful as 5,000 to 20,000 IE can be transplanted/kg recipient body weight. One IE can include from about 600 to about 8,600 cells. The cellular composition of islet preparations can be assessed using standard immunoassay methods. Antibodies that have binding affinity for insulin, glucagon, somatostatin, pancreatic polypeptide, amylase, and cytokeratin 19 can be used to identify .beta.-, .alpha.-, .delta.-, pp-, acinar, and ductal cells, respectively. Such antibodies are commercially available, e.g., from DAKO, Carpinteria, Calif. or Sigma Chemical Co., St. Louis, Mo. Binding can be detected by labeling, either directly or indirectly, the antibody having binding affinity for the particular protein (e.g., insulin) or a secondary antibody that binds to such an antibody. Suitable labels include, without limitation, radionuclides (e.g., ¹²⁵I, ¹³¹I, ³⁵S, ³H, ³²P, ³³P or ¹⁴C), fluorescent moieties (e.g., fluorescein, FITC, PerCP, rhodamine, or PE), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). Antibodies can be indirectly labeled by conjugation with biotin then detected with avidin or streptavidin labeled with a molecule described above. Methods of detecting or quantifying a label depend on the nature of the label and are known in the art. Examples of detectors include, without limitation, x-ray film, radioactivity counters, scintillation counters, spectrophotometers, colorimeters, fluorometers, luminometers, and densitometers. Immunological assays can be performed in a variety of known formats, including sandwich assays, competition assays (competitive RIA), or bridge immunoassays. See, for example, U.S. Pat. Nos. 5,296,347; 4,233,402; 4,098,876; and 4,034,074. The number of beta cells can be calculated based on the total DNA content and proportion of beta cells identified in the cellular composition sample. One IE can include from about 145 to 4,000 beta cells. Preparations of islet cells that contain at least 1×10⁶ beta cells/kg body weight of recipient (i.e., 4.5×10⁷ beta cells for a 45 kg recipient, 5×10⁷ beta cells for a 50 kg recipient, and 5.5×10⁷ beta cells for a 55 kg recipient) can be used. Preparations containing higher numbers of beta cells (e.g., at least 2×10⁶ beta cells/kg body weight of recipient, at least 3.5×10⁶ beta cells/kg body weight of recipient, or at least 5.0×10⁶ beta cells/kg body weight of recipient) are particularly useful. For example, preparations containing at least 3.5×10⁶ beta cells/kg body weight of recipient (i.e., about 1.58×10⁸ beta cells for a 45 kg recipient, about 1.75×10⁸ beta cells for a 50 kg recipient, and about 1.9×10⁸ beta cells for a 55 kg recipient) can sustain insulin independence for at least one year. Insulin content can be assessed using an immunoassay, e.g., the Human Insulin Enzyme Immunoassay (EIA) kit from Mercodia, Sweden, and corrected for the DNA content. Pico Green can be used to assess DNA content. In the Pico Green method, islet cells can be lysed with a solution containing ammonium hydroxide and a non-ionic detergent. Pico Green can be added to the sample and incubated in the dark. Samples are read on a fluorometer with an excitation of 480 nm and an emission of 520 nm and compared with a standard curve. Typically, one IE can include from about 4 to about 60 ng of DNA. Tissue volume of the preparation refers to the volume of the islet cell pellet before transplant. Islet cells can be collected in a pre-weighed tissue culture flask and the islets can be allowed to sediment to a bottom corner of the flask over a period of time (e.g., 5 minutes). The medium can be removed from the flask and the mass recorded. Suitable preparations of islet cells have a volume of 10 mL or less (e.g., 8 mL or less, 7.0 mL or less, 5 mL or less, 3 mL or less, or 2 mL or less). ATP content of islet cell preparations can be assessed via high performance liquid chromatography (HPLC) or by using an immunoassay (e.g., an ATP Determination Kit from Invitrogen Corp., Carlsbad, Calif.). In either method, samples can be prepared using the methods of Micheli et al. Clin. Chem. Acta 1993, 220:1-17 in which trichloroacetic acid is used to extract the ATP and a freon/amine solution is used to neutralize the sample. Preparations of islet cells that have at least 76 pmol ATP/.mu·g DNA (e.g., at least 80, 90, 100, 110, 150, 175, 190, or 193), as measured by HPLC, are particularly useful for transplants. A fluorescent dye inclusion/exclusion assay can be used to assess viability. See, for example, London et al., Hormone & Metabolic Research—Supplement 1990; 25:82-87. For example, fluorescein diacetate and propidium iodide (PI) can be used to assess viability. Fluorescein diacetate is dissociated by intracellular enzymes into free fluorescein, which fluoresces green under blue light excitation (490 nm) and provides evidence that the cells are alive and metabolically active. If the cell membrane has been damaged, PI can enter into the cell, intercalate into the nuclear DNA, and fluoresce red under green light excitation (545 nm). The proportion of green (viable) and red (dead) cells gives an indication of viability of the islet preparation. Alternatively, SYTO-13/ethidium bromide (SYTO/EB) and calcein AM/ethidium homodimer (C/EthD) fluorescent staining can be used to assess viability. See, for example, Barnett et al., Cell Transplant. 2004; 13(5):481-8. Preparations of islets that contain at least 70% (e.g., at least 75%, 80%, 85%, 90%, 95%, or 97%) viable cells are particularly useful for transplants.

The percent of IE recovered after culture can be determined using DTZ as described above. Preparations of islets in which at least 70% (e.g., 75%, 80%, 85%, 90%, or 95%) of the IE were recovered after culture are particularly useful for transplants. The percent necrotic and apoptotic cells can be assessed using known methods. For example, apoptosis can be assessed by examining DNA fragmentation. For example, a Cell Death Detection ELISA's (Roche Biochemicals, Indianapolis, Ind.) can be used to detect cytoplasmic histone-associated DNA fragments. Preparations of islets in which 30% or less (e.g., 25%, 20%, 15%, 10%, 5%, or less) of the cells are apoptotic or necrotic are useful for transplants. Glucose-stimulated insulin release is a measure of the functional capacity of the preparation. Standard techniques for static incubation and assessment of insulin release corrected for DNA content are utilized to determine the functional capacity of the islets. Ricordi et al., Acta Diabetol. Lat. 1990; 27:185-195. A stimulation index is calculated by dividing insulin release at 16.7 mM glucose by insulin release at 1.7 mM glucose. Preparations of islets that have a stimulation index of >1 (e.g., >4, >7, >10, >14, >17, or >27) are particularly useful for transplants. OCR can be measured using an OCR chamber (e.g., from Instech Laboratories, Inc., Plymouth Meeting, Pa.). See, for example, Papas et al., Cell Transplant. 2003; 12: 177; Papas et al., Cell Transplant. 2003; 12: 176; and Papas et al., Cell Transplant. 2001; 10: 519. Preparations of islets having an OCR of greater than >75 nmol/min/mg DNA (e.g., greater than >100, >150, >200, or >230 nmol/min/mg DNA) are particularly useful for transplants. Islet cells can be transplanted into, for example, the portal vein of a patient using surgical techniques such as mini laparotomy or percutaneous trans-hepatic portal venous catheterization. Prior to transplant, patients can undergo induction immunosuppression using different therapy regimens. Patients also can undergo post-transplant immunosuppression regimens. For example, induction therapy can include treatment with rabbit antithymocyte globulin (RATG), daclizumab, and etanercept (i.e., soluble tumor necrosis factor (TNF) receptor). RATG is a potent induction agent and also interferes with leukocyte responses to chemotactic signals and inhibits the expression of integrins required for firm cellular adhesion. Selective inhibition of TNF-.alpha. in the peritransplant period may be able to promote reversal of diabetes after marginal-mass islet transplants. Post-transplant, the function of engrafted islets may be enhanced by replacing or minimizing tacrolimus at 1 month post-transplant. Another example of an induction therapy can include use of anti-CD3 mAb hOKT3.gamma.1 (Ala-Ala), which can inactivate autoreactive, primed, islet-directed T cells immediately post-transplant. Anti-CD3 mAb, hOKT3.gamma.1 (Ala-Ala), is a humanized antibody that retains the binding region of OKT3 but replaces the murine framework with human amino acids. In addition, the human IgG1 Fc is mutated to prevent binding to the Fc receptor (FcR). Clinically, this engineered antibody has proven effective in preserving residual beta-cell function in new-onset type 1 diabetes. In addition, the hOKT3.gamma.1 (Ala-Ala) reversed kidney graft rejection. This dual activity against both autoreactive and alloreactive T cell responses occurred with markedly fewer side effects, as compared with the parental OKT3 antibody.

The invention also provides means of using perinatal tissue derived MSCs as facilitators of islet engraftment. In one embodiment, said cells are injected intra-portally to exploit the naturally occurring mechanism of intra-portal tolerogenesis, which has been previously demonstrated by administration of various cells of immunogenic origin intra-portally [1]. In a preferred embodiment, MSCs are initially administered intra-portally or intra-arterially via the celiac and SMA axis to stimulate a tolerogenic event.

In one embodiment of the invention, MSCs of the same donor as the pancreatic allograft are administered intra-portally, intra-arterially via the celiac and SMA axis or systemically in order to induce a donor-specific tolerogenic event. In another embodiment, MSCs are generated from a donor and placed in an immunoisolatory chamber to induce tolerogenesis. In another embodiment, donor specific MSC s are generated and administered together with T regulatory cells which have primed with supernatant of the stimulated MSCs with interferon gamma.

In a specific embodiment, said MSCs are isolated to possess substantial homogeneity and to be highly of perinatal tissue origin. Differentiation is the process by which an unspecialized (“uncommitted”) or less specialized cell acquires the features of a specialized cell, such as a nerve cell or a muscle cell, for example. A differentiated cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e. which cells it came from and what cells it can give rise to. The lineage of a cell places the cell within a hereditary scheme of development and differentiation. Within the context of the current invention, mesenchymal stem cells of perinatal tissue origin are extracted, or isolated to possess perinatal tissue therapeutic efficacy, in part by selecting of stem cells that are primarily of perinatal tissue origin.

As used herein, the phrase differentiates into a mesodermal, ectodermal or endodermal lineage refers to a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, chondrogenic, cardiogenic, dermatogenic, hematopoetic, hemangiogenic, myogenic, nephrogenic, urogenitogenic, osteogenic, pericardiogenic, or stromal. Examples of cells that differentiate into ectodermal lineage include, but are not limited to epidermal cells, neurogenic cells, and neurogliagenic cells. Examples of cells that differentiate into endodermal lineage include, but are not limited to, pleurigenic cells, hepatogenic cells, cells that give rise to the lining of the intestine, and cells that give rise to pancreogenic and splanchogenic cells.

Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore, the number of population doublings of a culture is greater than the passage number. The expansion of cells (i.e., the number of population doublings) during the period between passaging depends on many factors, including but not limited to the seeding density, substrate, medium, growth conditions, and time between passaging.

A conditioned medium is a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium. In some embodiments, the invention teaches the use of conditioned media, or concentrated conditioned media, or exosomes isolated from conditioned media of MSC to promote tolerogenesis.

As used herein, the term Growth Medium generally refers to a medium sufficient for the culturing of umbilicus-derived cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetalbovine serum, Hyclone, Logan Utah), or platelet lysate, antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases, different growth media are used, or different supplementations are provided, and these are normally indicated in the text as supplementations to Growth Medium.

Also relating to the present invention, the term standard growth conditions, as used herein refers to culturing of cells at 37° C., in a standard atmosphere comprising 5% CO₂. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

“Mesenchymal stem cell” or “MSC” in some embodiments refers to cells that are: (1) adherent to plastic; (2) express CD73, CD90, and CD105 antigens, while being CD14, CD34, CD45, and HLA-DR negative; and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; or c) ability to induce a healing response, either directly, or through elicitation of endogenous host repair mechanisms. As used herein, “mesenchymal stromal cell” or mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may include cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs), perinatal cells.

Oct-4 (oct-3 in humans) is a transcription factor expressed in the pregastrulation embryo, early cleavage stage embryo, cells of the inner cell mass of the blastocyst, and embryonic carcinoma (“EC”) cells (Nichols, J. et al. (1998) Cell 95: 379-91), and is down-regulated when cells are induced to differentiate. The oct-4 gene (oct-3 in humans) is transcribed into at least two splice variants in humans, oct-3A and oct-3B. The oct-3B splice variant is found in many differentiated cells whereas the oct-3A splice variant (also previously designated oct-3/4) is reported to be specific for the undifferentiated embryonic stem cell. See Shimozaki et al. (2003) Development 130: 2505-12. Expression of oct-3/4 plays an important role in determining early steps in embryogenesis and differentiation. Oct-3/4, in combination with rox-1, causes transcriptional activation of the Zn-finger protein rex-1, which is also required for maintaining ES cells in an undifferentiated state (Rosfjord, E. and Rizzino, A. (1997) Biochem Biophys Res Commun 203: 1795-802; Ben-Shushan, E. et al. (1998) Mol Cell Biol 18: 1866-78). In some embodiments of the invention, mesenchymal stem cells are selected for perinatal tissue expression of OCT-4. In other embodiments, OCT-4 expression is used as a means of identifying cells for culture and expansion subsequent to exposure to various culture conditions.

In a presently preferred embodiment, the isolation procedure may utilize an enzymatic digestion process. Enzymes are used to dissociate tissue to extract cellular populations that are subsequently harvested and grown for isolation of perinatal tissue derived mesenchymal stem cells. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A non-exhaustive list of enzymes compatible herewith includes mucolytic enzyme activities, metalloproteases, neutral proteases, serine proteases (such as trypsin, chymotrypsin, or elastase), and deoxyribonucleases. Presently preferred are enzyme activities selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37° C. during the enzyme treatment of the dissociation step. Diluting the digest may also improve yields of cells as cells may be trapped within a viscous digest.

While the use of enzyme activities is presently preferred, it is not required for isolation methods as provided herein. Methods based on mechanical separation alone may be successful in isolating the instant cells from the perinatal tissue as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37° C., however the temperature may range from about 35° C. to 39° C. depending on the other culture conditions and desired use of the cells or culture.

Presently, preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10¹⁴ cells or more are provided. Preferred are those methods which derive cells that can double sufficiently to produce at least about 10¹⁴, 10×¹⁵, 10×¹⁶, or 10¹⁷ or more cells when seeded at from about 10×³ to about 10⁶ cells/cm² in culture. Preferably these cell numbers are produced within 80, 70, or 60 days or less. In one embodiment, tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD45, CD117, CD141, or HLA-DR,DP, DQ.

In another embodiment of the invention, mesenchymal stem cells (MSCs) are used to enhance tolerance of allogeneic/xenogeneic islets or autologous islets. MSCs may be used in an unmanipulated manner, or manipulated by culture conditions, or may be genetically manipulated. Genetic manipulation may involve augmentation of immune suppressive/immune modulatory aspects, and/or transfection with autoantigen. In the case of diabetes, said autoantigen would involve islet autoantigens such as GAD, ISLA-1, insulin, pro-insulin, NRP, or peptides thereof. In one embodiment, MSCs are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′ 10⁷ cells/ml. Subsequently, the cells are centrifuged at 900 g for approximately 30 min or a time period sufficient to achieve separation of mononuclear cells from debris and erythrocytes. Said cells are then washed with PBS and plated at a density of approximately 1′ 10⁶ cells per ml in 175 cm² tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′ 10⁶ per 175 cm². BM-MSC are subsequently transfected with autoantigen and/or immune regulatory genes. In some embodiments of the invention, transfection is accomplished by use of lentiviral vectors, said means to perform lentiviral mediated transfection are well-known in the art and discussed in the following references [87-93]. Some specific examples of lentiviral based transfection of genes into MSC include transfection of SDF-1 to promote stem cell homing [94], or growth factors such as FGF-18 [95, 96], HGF [97], akt [98], TRAIL [99-102], PGE-1 [103], NUR77 to enhance migration [104], BDNF [105], HIF-1 alpha [106], CCL2 [107], interferon beta [108], HLA-G to enhance immune suppressive activity [109], hTERT [110], cytosine deaminase [111], OCT-4 to reduce senescence [112, 113], BAMBI to reduce TGF expression [114], HO-1 for antiapoptosis [115], LIGHT [116], miR-126 to enhance angiogenesis [117, 118], bcl-2 to prevent apoptosis [119], telomerase and myocardin to induce cardiogenesis [120], CXCR4 to accelerate hematopoietic recovery and reduce renal allograft rejection [122], wnt11 [123], Islet-1 to promote pancreatic differentiation [124], IL-27 to reduce autoimmune disease [125], ACE-2 to reduce sepsis [126], CXCR4 to reduce liver failure [127, 128], and the HGF antagonist NK4 to reduce cancer [129].

Cell cultures are tested for sterility weekly, endotoxin by limulus amebocyte lysate test, and mycoplasma by DNA-fluorochrome stain.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA or TryplE, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG (H+L) antibody. Confluent MSC in 175 cm² flasks are washed with Tyrode's salt solution, incubated with medium 199 (M199) for 60 min, and detached with 0.05% trypsin-EDTA (Gibco). Cells from 10 flasks were detached at a time and MSCs are resuspended in 40 ml of M199+1% human serum albumin (HSA; American Red Cross, Washington DC, USA). MSCs harvested from each 10-flask set were stored for up to 4 h at 4° C. and combined at the end of the harvest. A total of 2-10′ 10 6 MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, IL, USA) freezing bags using a rate-controlled freezer at a final concentration of 10% DMSO (Research Industries, Salt Lake City, UT, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

The dose of perinatal tissueMSCs appropriate to be used in accordance with various embodiments of the invention, will depend on numerous factors. It may vary considerably for different circumstances. The parameters that will determine optimal doses of perinatal tissue MSCs to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the perinatal tissue MSCs are syngeneic, autologous, allogeneic, or xenogeneic; their potency (specific activity); the site and/or distribution that must be targeted for the perinatal tissue MSCs to be effective; and such characteristics of the site such as accessibility to perinatal tissue MSCs and/or engraftment of perinatal tissue MSC. Additional parameters include co-administration with perinatal tissue MSC of other factors (such as growth factors and cytokines). The optimal dose in a given situation also will take into consideration the way in which the cells are formulated, the way they are administered, and the degree to which the cells will be localized at the target sites following administration. Finally, the determination of optimal dosing necessarily will provide an effective dose that is neither below the threshold of maximal beneficial effect nor above the threshold where the deleterious effects associated with the dose of perinatal tissue MSCs outweighs the advantages of the increased dose.

The optimal dose of perinatal tissue MSCs for some embodiments will be in the range of doses used for autologous, mononuclear bone marrow transplantation. For fairly pure preparations of perinatal tissue MSC, optimal doses in various embodiments will range from 10⁴ to 10⁸ perinatal tissue MSCs cells/kg of recipient mass per administration. In some embodiments, the optimal dose per administration will be between 10⁵ to 10⁷ perinatal tissue MSCs cells/kg. In many embodiments, the optimal dose per administration will be 5×10⁵ to 5×10⁶ perinatal tissue MSCs cells/kg. By way of reference, higher doses in the foregoing are analogous to the doses of nucleated cells used in autologous mononuclear bone marrow transplantation. Similarly, perinatal MSCs have been administered in doses of 100M to 400M in patients with no toxicity issues.

It is to be appreciated that a single dose may be delivered all at once, fractionally, or continuously over a period of time. The entire dose also may be delivered to a single location or spread fractionally over several locations.

In various embodiments, perinatal tissue MSCs may be administered in an initial dose, and thereafter maintained by further administration of perinatal tissue MSCs. Perinatal tissue MSCs may be administered by one method initially, and thereafter administered by the same method or one or more different methods. The subject's perinatal tissue MSC levels can be maintained by the ongoing administration of the cells. Various embodiments administer the perinatal tissue MSCs either initially or to maintain their level in the subject or both by intravenous injection. In a variety of embodiments, other forms of administration, are used, dependent upon the patient's condition and other factors, discussed elsewhere herein.

It is noted that human subjects are treated generally longer than experimental animals, but treatment generally has a length proportional to the length of the disease process and the effectiveness of the treatment. Those skilled in the art will take this into account in using the results of other procedures carried out in humans and/or in animals, such as rats, mice, non-human primates, and the like, to determine appropriate doses for humans. Such determinations, based on these considerations and taking into account guidance provided by the present disclosure and the prior art will enable the skilled artisan to do so without undue experimentation.

Suitable regimens for initial administration and further doses or for sequential administrations may all be the same or may be variable. Appropriate regiments can be ascertained by the skilled artisan, from this disclosure, the documents cited herein, and the knowledge in the art. The dose, frequency, and duration of treatment will depend on many factors, including the nature of the disease, the subject, and other therapies that may be administered. Accordingly, a wide variety of regimens may be used to administer perinatal tissue MSCs.

Perinatal tissue MSCs may be administered in many frequencies over a wide range of times. In some embodiments, perinatal tissue MSCs are administered over a period of less than one day. In other embodiments, they are administered over two, three, four, five, or six days. In some embodiments, perinatal tissue MSCs are administered one or more times per week, over a period of weeks. In other embodiments they are administered over a period of weeks for one to several months. In various embodiments, they may be administered over a period of months. In others they may be administered over a period of one or more years. Generally, lengths of treatment will be proportional to the length of the disease process, the effectiveness of the therapies being applied, and the condition and response of the subject being treated.

The MSCs administered in an environment that is already protolerogenic enhances said tolerogenicity, however for the practice of the invention, the augmentation of tolerance is supported by the administration of augmented T regulatory cells, which provide not only direct angiogenic support, but also act as tolerogenic and immunomodulatory cells that decrease rejection. For the practice of the invention, we describe below different ways in which MSCs modulate the immune system such that practitioners of the invention may utilize known means in the art. For example, MSCs modulate dendrite cell activity. Dendritic cells (DC) are considered the primary sentinels of the immune response, playing a key role in determining whether productive immunity will ensure, versus stimulation of T regulatory cells and suppression of immunity [2, 3]. Although various subtypes of DC exist, with varying specialized functions, one of the common themes appears to be that immature myeloid type DC reside in an immature state in the periphery, which engulf antigens and present in a tolerogenic manner to T cells in the lymph nodes. This is one of the mechanisms by which self-tolerance is maintained. Specifically, although small numbers of autoreactive T cells escape the thymic selection process, these T cells are either anergized, or their activity suppressed by T regulatory cells generated as a result of immature dendritic cells presenting self-antigens to autoreactive T cells. In contrast, in the presence of “danger” signals, such as toll like receptor agonists, immature DC take a mature phenotype, characterized by high expression of costimulatory molecules, and subsequently induce T cell activation [4-6]. In the context of T1D it has previously been demonstrated that targeting of diabetogenic autoantigens to immature DC leads to prevention of disease [7]. Administration of immature DC into 10 T1D patients resulted in increased C-peptide levels with some evidence of immunomodulatory activity [8].

Given the fundamental role of the DC in controlling immunity versus tolerance, the manipulation of DC maturation by MSC would strongly support an immune modulatory role of MSC. Early studies suggested that MSC may inhibit the ability of DC to stimulate CD4 and CD8 cells using in vitro systems, however, it was demonstrated that MSC also inhibited T cell activation directly [9]. Subsequently, Zhang et al. performed a definitive study in which bone marrow MSCs were cultured directly with monocytes which were stimulated to differentiate into DC using a standard IL-4 and GM-CSF protocol in the murine system. It was found that MSCs inhibit the up-regulation of CD1a, CD40, CD80, CD86, and HLA-DR during DC differentiation and prevent an increase of CD40, CD86, and CD83 expression during DC maturation. MSC supernatants had no effect on DCs differentiation, but they inhibited the up-regulation of CD83 during maturation. Both MSCs and their supernatants interfered with endocytosis of DCs, decreased their capacity to secret IL-12 and activate alloreactive T cells [10]. Using the human system, Aggarwal et al. cultured Prochymal BM derived MSCs together with DCs that were polarized to generate Th1 promoting cytokines (DC1) and DC polarized to generate Th2 cytokines (DC2). MSCs were demonstrated to inhibit production of TNF-alpha and IL-12 in DC1 cells while increase production of IL-10 in DC2 cells [10]. The concept of MSCs inducing immaturity in DCs was further demonstrated in mixed lymphocyte reactions where it was shown that addition of MSC would suppress MLR only in the presence of antigen presenting cells [11]. Interestingly, the addition of DC maturation agents such as LPS or antiCD40 antibody was capable of overcoming MSC mediated suppression, thus implying that inhibition of DC immunogenic activities by MSCs is a reversible process. The general ability of MSCs to suppress DC immune stimulatory activities, through inhibition of maturation and costimulatory molecule expression was replicated in other studies [12-17]. Other methods by which MSCs inhibit DC activity include blocking the physical interaction with T cells [12], blocking DC progenitor entry into cell cycle [12], production of TSG-6 [18], induction of Notch in DC progenitors [19], and arresting DC migration into lymph nodes in vivo [20-22]. In some aspects of the invention, immature DCs are administered to promote tolerogenesis. In other aspects, immature DCs are pulsed with antigens found on insulin producing cells.

Some of the immune suppressive effects of MSCs appear to be inducible by the presence of local inflammation. For example, a recent study showed that TLR activation on MSCs increases the ability of the MSCs to suppress T cell activation through blockade of DC maturation [23]. Other studies have shown that treatment of MSCs with inflammatory mediators such as IL-1 beta actually stimulates production of cytokines such as IL-10 that block DC maturation. IL-1 treated MSCs possess superior in vivo ability to suppress inflammatory diseases such as DSS induced colitis [24]. Similar augmentation of anti-inflammatory activity of MSC by pretreatment with inflammatory cytokines was also reported by treatment with IFN-gamma [25-27]. On a cellular level it has been reported that coculture of MSC with monocytes leads to enhanced immune suppressive activities of the MSC, in part through monocyte produced IL-1 [28].

Inhibition of T cell reactivity by MSCs has been widely described. One of the initial publications supporting this assessed baboon MSCs in vitro for their ability to elicit a proliferative response from allogeneic lymphocytes, to inhibit an ongoing allogeneic response, and to inhibit a proliferative response to potent T-cell mitogens. It was found that the MSCs failed to elicit a proliferative response from allogeneic lymphocytes. MSCs added into a mixed lymphocyte reaction, either on day 0 or on day 3, or to mitogen-stimulated lymphocytes, led to a greater than 50% reduction in proliferative activity. This effect could be maximized by escalating the dose of MSCs and could be reduced with the addition of exogenous IL-2. In vivo administration of MSCs led to prolonged skin graft survival when compared to control animals [29, 30]. Inhibition of T cell proliferation could not be restored by co-stimulation or pretreatment of the MSCs with IFN-gamma [31], which is intriguing given that the previous study mentioned showed IL-2 could overcome MSC mediated suppression. In vivo studies using humanized mice demonstrated that human MSCs were capable of suppressing human T cell responses in vivo, both allogenic and antigen-specific responses [32]. Inhibition of T cell activity seems to be not limited to proliferation but also was demonstrated to include suppression of cytotoxic activity of CD8 T cells [33, 34].

Several mechanisms have been reported for MSC suppression of T cell activation including inhibition of IL-2 receptor alpha (CD25) [35], induction of division arrest [36, 37], induction of T cell anergy directly or via immature DC [17], stimulation of apoptosis of activated T cells [39, 40], blockade of IL-2 signaling and induction of PGE2 production [41-46], induction of TGF-beta[47], production of HLA-G [48], expression of serine protease inhibitor 6 [49], stimulation of nitric oxide release [50-52], stimulation of indolamine 2,3 deoxygenase [53-56], expression of adenosine generating ectoenzymes such as CD39 and CD73 [57, 58], Galectin expression[59, 60], induction of hemoxygenase 1[61, 62], activation of the PD1 pathway [59, 63-65], Fas ligand expression [66, 67], CD200 expression [68], Th2 deviation [69-71], inhibition of Th17 differentiation [72-76], TSG-6 expression [77], NOTCH-1 expression [78], stimulation of Treg cell generation [79-86].

In another embodiment of the invention, the protection of the pancreatic islets (cells/tissues/organs) are facilitated by infusion of augmented T regulatory cells. The T regulatory cells can be administered before the islets to induce a tolerogenic process. In another embodiment, they are administered after the islets are transplanted to help decrease rejection episodes. This process may involve one or more doses of the augmented T regulatory cells.

The invention, in some embodiments, teaches the utilization of T regulatory cells or regenerative cells, whereby said cells possess immune regulatory and repair properties. It is known that a cardinal feature of the immune system allows for recognition and elimination of pathological threats, while selectively ignoring antigens that belong to the body. Traditionally, autoimmune conditions or conditions associated with cytokine storm, or allograft rejection are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia.

The current approach described teaches the generation of T cells and T regulatory cells which possess immunological and regenerative properties.

The invention provides novel stem cell types, methods of manufacture, and therapeutic uses. Provided are means of deriving stem cells possessing regenerative, immune modulatory, anti-inflammatory, and angiogenic/neurogenic activity from umbilical cord tissue such as perinatal tissue. In some embodiments, manipulation of stem cell “potency” is disclosed through hypoxic manipulation, growth on non-xenogeneic conditions, as well as addition of epigenetic modulators.

The cells of the invention are cultured under hypoxia, in one embodiment, cultured in order to induce and/or augment expression of chemokine receptors. One such receptor is CXCR-4. The population of cells, including population of umbilical cord mesenchymal cells, may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition, or alternatively, <1%, <2%, <3%, <4%, <5%, <6%, <7%, <8%, <9%, or <10% of the population of cells may express CD14 and/or CD45. The perinatal cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof.

The T regulatory cells which are regenerative are derived by obtaining a peripheral blood mononuclear cells via an apheresis from a patient. The fresh or frozen leuko-pack is enriched, depleted and separated for a group of factors such as FOXP3, CD4, CD8, CD25, and/or CD39 using antibody bead based and density gradient optimization. The augmentation of the T cells is performed via either directed co-culture of the perinatal tissue MSCs which have been stimulated with one of many factors such as gamma interferon. The supernatant of the stimulated perinatal tissue MSCs and/or the stimulated perinatal cells are co-cultured with the T cells to produce the augmented T regulatory cells which have better immunomodulatory and anti-rejection properties compared to standard T cells. The cells may also be derived from IPS cells to enable larger scalability. These augmented T regulatory cells are then administered to the patient before, during, and/or after the pancreatic islet transplantation in doses to 10M to 250M cells IV. The augmented T cells may also be given at times of rejection to help immunomodulate and reduce the loss of the pancreatic islets/tissues/organ. This will allow for repeat dosing to best optimize the patient's immunosuppressive regimen.

Overall, the utilization of the regenerative cells in combination or stand-alone before and/or after have a beneficial impact on either improvement engraftment and survival of pancreatic cells, tissue, and/or the organ and reduces the impact of ischemia, rejection, and improved tolerance and can be applied more widely to other tissues and organs.

### REFERENCES

1. He, F., et al., Increased CD4+CD25+Foxp3+ regulatory T cells in tolerance induced by portal venous injection. Surgery, 2009. 145(6): p. 663-74.
2. Steinman, R. M., D. Hawiger, and M. C. Nussenzweig, Tolerogenic dendritic cells. Annu Rev Immunol, 2003. 21: p. 685-711.
3. Adema, G. J., Dendritic cells from bench to bedside and back. Immunol Lett, 2009. 122(2): p. 128-30.
4. Steinman, R. M. and K. Inaba, Myeloid dendritic cells. J Leukoc Biol, 1999. 66(2): p. 205-8.
5. Steinman, R. M., Dendritic cells and the control of immunity: enhancing the efficiency of antigen presentation. Mt Sinai J Med, 2001. 68(3): p. 160-6.
6. Bonifaz, L., et al., Efficient targeting of protein antigen to the dendritic cell receptor DEC-205 in the steady state leads to antigen presentation on major histocompatibility complex class I products and peripheral CD8+ T cell tolerance. J Exp Med, 2002. 196(12): p. 1627-38.
7. Mukhopadhaya, A., et al., Selective delivery of beta cell antigen to dendritic cells in vivo leads to deletion and tolerance of autoreactive CD8+ T cells in NOD mice. Proc Natl Acad Sci USA, 2008. 105(17): p. 6374-9.
8. Giannoukakis, N., et al., Phase I (safety) study of autologous tolerogenic dendritic cells in type 1 diabetic patients. Diabetes Care, 2011. 34(9): p. 2026-32.
9. Krampera, M., et al., Bone marrow mesenchymal stem cells inhibit the response of naive and memory antigen-specific T cells to their cognate peptide. Blood, 2003. 101(9): p. 3722-9.
10. Zhang, W., et al., Effects of mesenchymal stem cells on differentiation, maturation, and function of human monocyte-derived dendritic cells. Stem Cells Dev, 2004. 13(3): p. 263-71.
11. Beyth, S., et al., Human mesenchymal stem cells alter antigen-presenting cell maturation and induce T-cell unresponsiveness. Blood, 2005. 105(5): p. 2214-9.
12. Jiang, X. X., et al., Human mesenchymal stem cells inhibit differentiation and function of monocyte-derived dendritic cells. Blood, 2005. 105(10): p. 4120-6.
13. Fibbe, W. E., A. J. Nauta, and H. Roelofs, Modulation of immune responses by mesenchymal stem cells. Ann N Y Acad Sci, 2007. 1106: p. 272-8.
14. Djouad, F., et al., Mesenchymal stem cells inhibit the differentiation of dendritic cells through an interleukin-6-dependent mechanism. Stem Cells, 2007. 25(8): p. 2025-32.
15. Jung, Y. J., et al., MSC-DC interactions: MSC inhibit maturation and migration of BM-derived DC. Cytotherapy, 2007. 9(5): p. 451-8.
16. Chen, L., et al., Effects of human mesenchymal stem cells on the differentiation of dendritic cells from CD34+ cells. Stem Cells Dev, 2007. 16(5): p. 719-31.
17. Wang, Q., et al., Murine bone marrow mesenchymal stem cells cause mature dendritic cells to promote T-cell tolerance. Scand J Immunol, 2008. 68(6): p. 607-15.
18. Oh, J. Y., et al., Intravenous mesenchymal stem cells prevented rejection of allogeneic corneal transplants by aborting the early inflammatory response. Mol Ther, 2012. 20(11): p. 2143-52.
19. Li, Y. P., et al., Human mesenchymal stem cells license adult CD34+ hemopoietic progenitor cells to differentiate into regulatory dendritic cells through activation of the Notch pathway. J Immunol, 2008. 180(3): p. 1598-608.
20. Chiesa, S., et al., Mesenchymal stem cells impair in vivo T-cell priming by dendritic cells. Proc Natl Acad Sci USA, 2011. 108(42): p. 17384-9.
21. English, K., F. P. Barry, and B. P. Mahon, Murine mesenchymal stem cells suppress dendritic cell migration, maturation and antigen presentation. Immunol Lett, 2008. 115(1): p. 50-8.
22. Li, H., et al., Mesenchymal stem cells alter migratory property of T and dendritic cells to delay the development of murine lethal acute graft-versus-host disease. Stem Cells, 2008. 26(10): p. 2531-41.
23. Opitz, C. A., et al., Toll-like receptor engagement enhances the immunosuppressive properties of human bone marrow-derived mesenchymal stem cells by inducing indoleamine-2,3-dioxygenase-1 via interferon-beta and protein kinase R. Stem Cells, 2009. 27(4): p. 909-19.
24. Fan, H., et al., Pre-treatment with IL-1beta enhances the efficacy of MSC transplantation in DSS-induced colitis. Cell Mol Immunol, 2012. 9(6): p. 473-81.
25. Duijvestein, M., et al., Pretreatment with interferon-gamma enhances the therapeutic activity of mesenchymal stromal cells in animal models of colitis. Stem Cells, 2011. 29(10): p. 1549-58.
26. Krampera, M., et al., Role for interferon-gamma in the immunomodulatory activity of human bone marrow mesenchymal stem cells. Stem Cells, 2006. 24(2): p. 386-98.
27. Polchert, D., et al., IFN-gamma activation of mesenchymal stem cells for treatment and prevention of graft versus host disease. Eur J Immunol, 2008. 38(6): p. 1745-55.
28. Groh, M. E., et al., Human mesenchymal stem cells require monocyte-mediated activation to suppress alloreactive T cells. Exp Hematol, 2005. 33(8): p. 928-34.
29. Bartholomew, A., et al., Mesenchymal stem cells suppress lymphocyte proliferation in vitro and prolong skin graft survival in vivo. Exp Hematol, 2002. 30(1): p. 42-8.
30. Beggs, K. J., et al., Immunologic consequences of multiple, high-dose administration of allogeneic mesenchymal stem cells to baboons. Cell Transplant, 2006. 15(8-9): p. 711-21.
31. Tse, W. T., et al., Suppression of allogeneic T-cell proliferation by human marrow stromal cells: implications in transplantation. Transplantation, 2003. 75(3): p. 389-97.
32. Maitra, B., et al., Human mesenchymal stem cells support unrelated donor hematopoietic stem cells and suppress T-cell activation. Bone Marrow Transplant, 2004. 33(6): p. 597-604.
33. Rasmusson, I., et al., Mesenchymal stem cells inhibit the formation of cytotoxic T lymphocytes, but not activated cytotoxic T lymphocytes or natural killer cells. Transplantation, 2003. 76(8): p. 1208-13.
34. Angoulvant, D., et al., Human mesenchymal stem cells suppress induction of cytotoxic response to alloantigens. Biorheology, 2004. 41(3-4): p. 469-76.
35. Le Blanc, K., et al., Mesenchymal stem cells inhibit the expression of CD25 (interleukin-2 receptor) and CD38 on phytohaemagglutinin-activated lymphocytes. Scand J Immunol, 2004. 60(3): p. 307-15.
36. Glennie, S., et al., Bone marrow mesenchymal stem cells induce division arrest anergy of activated T cells. Blood, 2005. 105(7): p. 2821-7.
37. Kim, J. A., et al., The inhibition of T-cells proliferation by mouse mesenchymal stem cells through the induction of p16INK4A-cyclin D1/cdk4 and p21 waft, p27kip1-cyclin E/cdk2 pathways. Cell Immunol, 2007. 245(1): p. 16-23.
38. Zappia, E., et al., Mesenchymal stem cells ameliorate experimental autoimmune encephalomyelitis inducing T-cell anergy. Blood, 2005. 106(5): p. 1755-61.
39. Plumas, J., et al., Mesenchymal stem cells induce apoptosis of activated T cells. Leukemia, 2005. 19(9): p. 1597-604.
40. Lim, J. H., et al., Immunomodulation of delayed-type hypersensitivity responses by mesenchymal stem cells is associated with bystander T cell apoptosis in the draining lymph node. J Immunol, 2010. 185(7): p. 4022-9.
41. Rasmusson, I., et al., Mesenchymal stem cells inhibit lymphocyte proliferation by mitogens and alloantigens by different mechanisms. Exp Cell Res, 2005. 305(1): p. 33-41.
42. Xu, G., et al., Immunosuppressive properties of cloned bone marrow mesenchymal stem cells. Cell Res, 2007. 17(3): p. 240-8.
43. English, K., et al., Cell contact, prostaglandin E(2) and transforming growth factor beta 1 play non-redundant roles in human mesenchymal stem cell induction of CD4+CD25(High) forkhead box P3+ regulatory T cells. Clin Exp Immunol, 2009. 156(1): p. 149-60.
44. Spaggiari, G. M., et al., MSCs inhibit monocyte-derived DC maturation and function by selectively interfering with the generation of immature DCs: central role of MSC-derived prostaglandin E2. Blood, 2009. 113(26): p. 6576-83.
45. Yanez, R., et al., Prostaglandin E2 plays a key role in the immunosuppressive properties of adipose and bone marrow tissue-derived mesenchymal stromal cells. Exp Cell Res, 2010. 316(19): p. 3109-23.
46. Zafranskaya, M., et al., PGE2 Contributes to In vitro MSC-Mediated Inhibition of Non-Specific and Antigen-Specific T Cell Proliferation in MS Patients. Scand J Immunol, 2013. 78(5): p. 455-62.
47. Nasef, A., et al., Identification of IL-10 and TGF-beta transcripts involved in the inhibition of T-lymphocyte proliferation during cell contact with human mesenchymal stem cells. Gene Expr, 2007. 13(4-5): p. 217-26.
48. Magatti, M., et al., Human amnion mesenchyme harbors cells with allogeneic T-cell suppression and stimulation capabilities. Stem Cells, 2008. 26(1): p. 182-92.
49. El Haddad, N., et al., Mesenchymal stem cells express serine protease inhibitor to evade the host immune response. Blood, 2011. 117(4): p. 1176-83.
50. Sato, K., et al., Nitric oxide plays a critical role in suppression of T-cell proliferation by mesenchymal stem cells. Blood, 2007. 109(1): p. 228-34.
51. Oh, I., et al., Interferon-gamma and NF-kappaB mediate nitric oxide production by mesenchymal stromal cells. Biochem Biophys Res Commun, 2007. 355(4): p. 956-62.
52. Ren, G., et al., Mesenchymal stem cell-mediated immunosuppression occurs via concerted action of chemokines and nitric oxide. Cell Stem Cell, 2008. 2(2): p. 141-50.
53. DelaRosa, O., et al., Requirement of IFN-gamma-mediated indoleamine 2,3-dioxygenase expression in the modulation of lymphocyte proliferation by human adipose-derived stem cells. Tissue Eng Part A, 2009. 15(10): p. 2795-806.
54. Tipnis, S., C. Viswanathan, and A. S. Majumdar, Immunosuppressive properties of human umbilical cord-derived mesenchymal stem cells: role of B7-H1 and IDO. Immunol Cell Biol, 2010. 88(8): p. 795-806.
55. Ge, W., et al., Regulatory T-cell generation and kidney allograft tolerance induced by mesenchymal stem cells associated with indoleamine 2,3-dioxygenase expression. Transplantation, 2010. 90(12): p. 1312-20.
56. Francois, M., et al., Human MSC suppression correlates with cytokine induction of indoleamine 2,3-dioxygenase and bystander M2 macrophage differentiation. Mol Ther, 2012. 20(1): p. 187-95.
57. Sattler, C., et al., Inhibition of T-cell proliferation by murine multipotent mesenchymal stromal cells is mediated by CD39 expression and adenosine generation. Cell Transplant, 2011. 20(8): p. 1221-30.
58. Saldanha-Araujo, F., et al., Mesenchymal stromal cells up-regulate CD39 and increase adenosine production to suppress activated T-lymphocytes. Stem Cell Res, 2011. 7(1): p. 66-74.
59. Xue, Q., et al., The negative co-signaling molecule b7-h4 is expressed by human bone marrow-derived mesenchymal stem cells and mediates its T-cell modulatory activity. Stem Cells Dev, 2010. 19(1): p. 27-38.
60. Gieseke, F., et al., Human multipotent mesenchymal stromal cells use galectin-1 to inhibit immune effector cells. Blood, 2010. 116(19): p. 3770-9.
61. Chabannes, D., et al., A role for heme oxygenase-1 in the immunosuppressive effect of adult rat and human mesenchymal stem cells. Blood, 2007. 110(10): p. 3691-4.
62. Mougiakakos, D., et al., The impact of inflammatory licensing on heme oxygenase-1-mediated induction of regulatory T cells by human mesenchymal stem cells. Blood, 2011. 117(18): p. 4826-35.
63. Augello, A., et al., Bone marrow mesenchymal progenitor cells inhibit lymphocyte proliferation by activation of the programmed death 1 pathway. Eur J Immunol, 2005. 35(5): p. 1482-90.
64. Sheng, H., et al., A critical role of IFNgamma in priming MSC-mediated suppression of T cell proliferation through up-regulation of B7-H1. Cell Res, 2008. 18(8): p. 846-57.
65. Luz-Crawford, P., et al., Mesenchymal stem cells repress Th17 molecular program through the PD-1 pathway. PLoS One, 2012. 7(9): p. e45272.
66. Akiyama, K., et al., Mesenchymal-stem-cell-induced immunoregulation involves FAS-ligand-/FAS-mediated T cell apoptosis. Cell Stem Cell, 2012. 10(5): p. 544-55.
67. Gu, Y. Z., et al., Different roles of PD-L1 and FasL in immunomodulation mediated by human placenta-derived mesenchymal stem cells. Hum Immunol, 2013. 74(3): p. 267-76.
68. Najar, M., et al., Characterization and functionality of the CD200-CD200R system during mesenchymal stromal cell interactions with T-lymphocytes. Immunol Lett, 2012. 146(1-2): p. 50-6.
69. Batten, P., et al., Human mesenchymal stem cells induce T cell anergy and downregulate T cell allo-responses via the TH2 pathway: relevance to tissue engineering human heart valves. Tissue Eng, 2006. 12(8): p. 2263-73.
70. Lu, X., et al., Immunomodulatory effects of mesenchymal stem cells involved in favoring type 2 T cell subsets. Transpl Immunol, 2009. 22(1-2): p. 55-61.
71. Zanone, M. M., et al., Human mesenchymal stem cells modulate cellular immune response to islet antigen glutamic acid decarboxylase in type 1 diabetes. J Clin Endocrinol Metab, 2010. 95(8): p. 3788-97.
72. Ko, E., et al., Mesenchymal stem cells inhibit the differentiation of CD4+ T cells into interleukin-17-secreting T cells. Acta Haematol, 2008. 120(3): p. 165-7.
73. Rafei, M., et al., Mesenchymal stromal cells ameliorate experimental autoimmune encephalomyelitis by inhibiting CD4 Th17 T cells in a CC chemokine ligand 2-dependent manner. J Immunol, 2009. 182(10): p. 5994-6002.
74. Tatara, R., et al., Mesenchymal stromal cells inhibit Th17 but not regulatory T-cell differentiation. Cytotherapy, 2011. 13(6): p. 686-94.
75. Duffy, M. M., et al., Mesenchymal stem cell inhibition of T-helper 17 cell-differentiation is triggered by cell-cell contact and mediated by prostaglandin E2 via the EP4 receptor. Eur J Immunol, 2011. 41(10): p. 2840-51.
76. Luz-Crawford, P., et al., Mesenchymal stem cells generate a CD4+CD25+Foxp3+ regulatory T cell population during the differentiation process of Th1 and Th17 cells. Stem Cell Res Ther, 2013. 4(3): p. 65.
77. Kota, D. J., et al., TSG-6 produced by hMSCs delays the onset of autoimmune diabetes by suppressing Th1 development and enhancing tolerogenicity. Diabetes, 2013. 62(6): p. 2048-58.
78. Del Papa, B., et al., Notch1 modulates mesenchymal stem cells mediated regulatory T-cell induction. Eur J Immunol, 2013. 43(1): p. 182-7.
79. Maccario, R., et al., Interaction of human mesenchymal stem cells with cells involved in alloantigen-specific immune response favors the differentiation of CD4+ T-cell subsets expressing a regulatory/suppressive phenotype. Haematologica, 2005. 90(4): p. 516-25.
80. Prevosto, C., et al., Generation of CD4+ or CD8+ regulatory T cells upon mesenchymal stem cell-lymphocyte interaction. Haematologica, 2007. 92(7): p. 881-8.
81. Di Ianni, M., et al., Mesenchymal cells recruit and regulate T regulatory cells. Exp Hematol, 2008. 36(3): p. 309-18.
82. Casiraghi, F., et al., Pretransplant infusion of mesenchymal stem cells prolongs the survival of a semiallogeneic heart transplant through the generation of regulatory T cells. J Immunol, 2008. 181(6): p. 3933-46.
83. Boumaza, I., et al., Autologous bone marrow-derived rat mesenchymal stem cells promote PDX-1 and insulin expression in the islets, alter T cell cytokine pattern and preserve regulatory T cells in the periphery and induce sustained normoglycemia. J Autoimmun, 2009. 32(1): p. 33-42.
84. Ye, Z., et al., Immunosuppressive effects of rat mesenchymal stem cells: involvement of CD4+CD25+ regulatory T cells. Hepatobiliary Pancreat Dis Int, 2008. 7(6): p. 608-14.
85. Madec, A. M., et al., Mesenchymal stem cells protect NOD mice from diabetes by inducing regulatory T cells. Diabetologia, 2009. 52(7): p. 1391-9.
86. Melief, S. M., et al., Multipotent stromal cells induce human regulatory T cells through a novel pathway involving skewing of monocytes toward anti-inflammatory macrophages. Stem Cells, 2013. 31(9): p. 1980-91.
87. Zhang, X. Y., et al., Lentiviral vectors for sustained transgene expression in human bone marrow-derived stromal cells. Mol Ther, 2002. 5(5 Pt 1): p. 555-65.
88. Kyriakou, C. A., et al., Human mesenchymal stem cells (hMSCs) expressing truncated soluble vascular endothelial growth factor receptor (tsFlk-1) following lentiviral-mediated gene transfer inhibit growth of Burkitt's lymphoma in a murine model. J Gene Med, 2006. 8(3): p. 253-64.
89. Worsham, D. N., et al., In vivo gene transfer into adult stem cells in unconditioned mice by in situ delivery of a lentiviral vector. Mol Ther, 2006. 14(4): p. 514-24.
90. Rabin, N., et al., A new xenograft model of myeloma bone disease demonstrating the efficacy of human mesenchymal stem cells expressing osteoprotegerin by lentiviral gene transfer. Leukemia, 2007. 21(10): p. 2181-91.
91. Kallifatidis, G., et al., Improved lentiviral transduction of human mesenchymal stem cells for therapeutic intervention in pancreatic cancer. Cancer Gene Ther, 2008. 15(4): p. 231-40.
92. Meyerrose, T. E., et al., Lentiviral-transduced human mesenchymal stem cells persistently express therapeutic levels of enzyme in a xenotransplantation model of human disease. Stem Cells, 2008. 26(7): p. 1713-22.
93. McGinley, L., et al., Lentiviral vector mediated modification of mesenchymal stem cells & enhanced survival in an in vitro model of ischaemia. Stem Cell Res Ther, 2011. 2(2): p. 12.
94. Liang, X., et al., Human bone marrow mesenchymal stem cells expressing SDF-1 promote hematopoietic stem cell function of human mobilised peripheral blood CD34+ cells in vivo and in vitro. Int J Radiat Biol, 2010. 86(3): p. 230-7.
95. Hamidouche, Z., et al., Autocrine fibroblast growth factor 18 mediates dexamethasone-induced osteogenic differentiation of murine mesenchymal stem cells. J Cell Physiol, 2010. 224(2): p. 509-15.
96. Glavaski-Joksimovic, A., et al., Glial cell line-derived neurotrophic factor-secreting genetically modified human bone marrow-derived mesenchymal stem cells promote recovery in a rat model of Parkinson's disease. J Neurosci Res, 2010. 88(12): p. 2669-81.
97. Liu, A. M., et al., Umbilical cord-derived mesenchymal stem cells with forced expression of hepatocyte growth factor enhance remyelination and functional recovery in a rat intracerebral hemorrhage model. Neurosurgery, 2010. 67(2): p. 357-65; discussion 365-6.
98. Yu, Y. S., et al., AKT-modified autologous intracoronary mesenchymal stem cells prevent remodeling and repair in swine infarcted myocardium. Chin Med J (Engl), 2010. 123(13): p. 1702-8.
99. Mueller, L. P., et al., TRAIL-transduced multipotent mesenchymal stromal cells (TRAIL-MSC) overcome TRAIL resistance in selected CRC cell lines in vitro and in vivo. Cancer Gene Ther, 2011. 18(4): p. 229-39.
100. Yan, C., et al., Suppression of orthotopically implanted hepatocarcinoma in mice by umbilical cord-derived mesenchymal stem cells with sTRAIL gene expression driven by AFP promoter. Biomaterials, 2014. 35(9): p. 3035-43.
101. Deng, Q., et al., TRAIL-secreting mesenchymal stem cells promote apoptosis in heat-shock-treated liver cancer cells and inhibit tumor growth in nude mice. Gene Ther, 2014. 21(3): p. 317-27.
102. Sage, E. K., et al., Systemic but not topical TRAIL-expressing mesenchymal stem cells reduce tumour growth in malignant mesothelioma. Thorax, 2014. 69(7): p. 638-47.
103. Lian, W. S., et al., In vivo therapy of myocardial infarction with mesenchymal stem cells modified with prostaglandin I synthase gene improves cardiac performance in mice. Life Sci, 2011. 88(9-10): p. 455-64.
104. Maijenburg, M. W., et al., Nuclear receptors Nur77 and Nurr1 modulate mesenchymal stromal cell migration. Stem Cells Dev, 2012. 21(2): p. 228-38.
105. Harper, M. M., et al., Transplantation of BDNF-secreting mesenchymal stem cells provides neuroprotection in chronically hypertensive rat eyes. Invest Ophthalmol Vis Sci, 2011. 52(7): p. 4506-15.
106. Zou, D., et al., In vitro study of enhanced osteogenesis induced by HIF-1 alpha-transduced bone marrow stem cells. Cell Prolif, 2011. 44(3): p. 234-43.
107. Saito, S., et al., Mesenchymal stem cells stably transduced with a dominant-negative inhibitor of CCL2 greatly attenuate bleomycin-induced lung damage. Am J Pathol, 2011. 179(3): p. 1088-94.
108. Seo, K. W., et al., Anti-tumor effects of canine adipose tissue-derived mesenchymal stromal cell-based interferon-beta gene therapy and cisplatin in a mouse melanoma model. Cytotherapy, 2011. 13(8): p. 944-55.
109. Yang, H. M., et al., Enhancement of the immunosuppressive effect of human adipose tissue-derived mesenchymal stromal cells through HLA-GI expression. Cytotherapy, 2012. 14(1): p. 70-9.
110. Liang, X. J., et al., Differentiation of human umbilical cord mesenchymal stem cells into hepatocyte-like cells by hTERT gene transfection in vitro. Cell Biol Int, 2012. 36(2): p. 215-21.
111. Fei, S., et al., The antitumor effect of mesenchymal stem cells transduced with a lentiviral vector expressing cytosine deaminase in a rat glioma model. J Cancer Res Clin Oncol, 2012. 138(2): p. 347-57.
112. Jaganathan, B. G. and D. Bonnet, Human mesenchymal stromal cells senesce with exogenous OCT4. Cytotherapy, 2012. 14(9): p. 1054-63.
113. Han, S. H., et al., Effect of ectopic OCT4 expression on canine adipose tissue-derived mesenchymal stem cell proliferation. Cell Biol Int, 2014. 38(10): p. 1163-73.
114. Shangguan, L., et al., Inhibition of TGF-beta/Smad signaling by BAMBI blocks differentiation of human mesenchymal stem cells to carcinoma-associated fibroblasts and abolishes their protumor effects. Stem Cells, 2012. 30(12): p. 2810-9.
115. Kearns-Jonker, M., et al., Genetically Engineered Mesenchymal Stem Cells Influence Gene Expression in Donor Cardiomyocytes and the Recipient Heart. J Stem Cell Res Ther, 2012. S1.
116. Ma, G. L., et al., [Study of inhibiting and killing effects of transgenic LIGHT human umbilical cord blood mesenchymal stem cells on stomach cancer]. Zhonghua Wei Chang Wai Ke Za Zhi, 2012. 15(11): p. 1178-81.
117. Huang, F., et al., Mesenchymal stem cells modified with miR-126 release angiogenic factors and activate Notch ligand Delta-like-4, enhancing ischemic angiogenesis and cell survival. Int J Mol Med, 2013. 31(2): p. 484-92.
118. Huang, F., et al., Overexpression of miR-126 promotes the differentiation of mesenchymal stem cells toward endothelial cells via activation of PI3K/Akt and MAPK/ERK pathways and release of paracrine factors. Biol Chem, 2013. 394(9): p. 1223-33.
119. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.
120. Madonna, R., et al., Transplantation of mesenchymal cells rejuvenated by the overexpression of telomerase and myocardin promotes revascularization and tissue repair in a murine model of hindlimb ischemia. Circ Res, 2013. 113(7): p. 902-14.
121. Zang, Y., et al., [Influence of CXCR4 overexpressed mesenchymal stem cells on hematopoietic recovery of irradiated mice]. Zhongguo Shi Yan Xue Ye Xue Za Zhi, 2013. 21(5): p. 1261-5.
122. Cao, Z., et al., Protective effects of mesenchymal stem cells with CXCR4 up-regulation in a rat renal transplantation model. PLoS One, 2013. 8(12): p. e82949.
123. Liu, S., et al., Overexpression of Wnt11 promotes chondrogenic differentiation of bone marrow-derived mesenchymal stem cells in synergism with TGF-beta. Mol Cell Biochem, 2014. 390(1-2): p. 123-31.
124. Yin, N., et al., Islet-1 promotes the cardiac-specific differentiation of mesenchymal stem cells through the regulation of histone acetylation. Int J Mol Med, 2014. 33(5): p. 1075-82.
125. Hajizadeh-Sikaroodi, S., et al., Lentiviral Mediating Genetic Engineered Mesenchymal Stem Cells for Releasing IL-27 as a Gene Therapy Approach for Autoimmune Diseases. Cell J, 2014. 16(3): p. 255-62.
126. He, H., et al., Mesenchymal Stem Cells Overexpressing Angiotensin-Converting Enzyme 2 Rescue Lipopolysaccharide-Induced Lung Injury. Cell Transplant, 2014.
127. Ma, H. C., et al., Targeted migration of mesenchymal stem cells modified with CXCR4 to acute failing liver improves liver regeneration. World J Gastroenterol, 2014. 20(40): p. 14884-94.
128. Yang, J. X., et al., CXCR4 receptor overexpression in mesenchymal stem cells facilitates treatment of acute lung injury in rats. J Biol Chem, 2015. 290(4): p. 1994-2006.
129. Zhu, Y., et al., Mesenchymal stem cell-based NK4 gene therapy in nude mice bearing gastric cancer xenografts. Drug Des Devel Ther, 2014. 8: p. 2449-62.

1. A method of increasing efficacy of an islet cell transplant for treatment of diabetes, wherein said method comprises the steps of: a) obtaining a diabetic patient in need of an islet cell transplant; b) administering to said patient one or more tolerogenic regenerative antigen presenting cells; c) administering to said patient one or more T cells that have been exposed to a regenerative cell for a sufficient time period to allow said T cells to possess ability to enhance islet engraftment; and d) administering said islet cells to said patient.

2. The method of claim 1, wherein said regenerative cell has been in contact with said one or more T cells either in a cell to cell contact dependent manner or in contact through sharing of conditioned media.

3. The method of claim 2, wherein said regenerative cell is a mesenchymal stem cell derived from tissue selected from the group consisting of: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) perinatal tissue.

4. The method of claim 3, wherein said mesenchymal stem cell expresses a marker selected from the group consisting of: STRO-1, CD90, CD56, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

5. The method of claim 3, wherein said mesenchymal stem cell is generated from a pluripotent stem cell.

6. The method of claim 5, wherein said pluripotent stem cell is selected from the group consisting of: a) an embryonic stem cell; b) an inducible pluripotent stem cell; c) a parthenogenic stem cell; and d) a somatic cell nuclear transfer derived stem cell.

7. The method of claim 6, wherein said embryonic stem cell population expresses genes selected from the group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

8. The method of claim 6, wherein said inducible pluripotent stem cell possesses markers selected from the group consisting of: CD10, CD13, CD44, CD73, CD90, PDGFr-alpha, PD-L2, and HLA-A, -B, -C and possesses the ability to undergo at least 40 doublings in culture, while maintaining a normal karyotype upon passaging.

9. The method of claim 6, wherein said parthenogenic stem cells wherein said parthenogenically derived stem cells are generated by addition of a calcium flux inducing agent to activate an oocyte followed by enrichment of cells expressing markers selected from the group consisting of: SSEA-4, TRA 1-60 and TRA 1-81.

10. A method of preventing or treating rejection of allogenic and/or xenogenic cells implantation in a patient suffering from diabetes by administering regenerative cells combined with immunosuppressive agents to said patient.

11. The method of claim 10, wherein said regenerative cells are mesenchymal stem cells.

12. The method of claim 10, wherein said mesenchymal stem cells are derived from a source selected from the group consisting of: a) bone marrow; b) perivascular tissue; c) adipose tissue; d) perinatal tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; and v) skeletal muscle tissue.

13. The method of claim 10, wherein said tissue derived mesenchymal stem cells are isolated from tissues containing cells selected from the group consisting of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

14. The method of claim 12, wherein said mesenchymal stem cells express a marker selected from the group consisting of: a) CD73; b) CD90; and c) CD105.

15. The method of claim 12, wherein said mesenchymal stem cells from perinatal tissue express markers selected from the group consisting of: a) oxidized low density lipoprotein receptor 1; b) chemokine receptor ligand 3; and c) granulocyte chemotactic protein.

16. The method of claim 12, wherein said mesenchymal stem cells from perinatal tissue express, relative to a human fibroblast, increased levels of interleukin 8 and reticulon 1.

17. The method of claim 1, wherein said tolerogenic cells are monocytes.

18. The method of claim 1, wherein said monocytes are capable of inducing generation of T regulatory (Treg) cells.

19. The method of claim 18, wherein said Treg cells are capable of suppressing rejection of allogeneic islet cells.

20. The method of claim 18, wherein said Treg cells are capable of suppressing dendritic cell maturation.
